(19) United States
(12) Patent Application Publication
(10) Pub. No.: US 2025/0213425 A1
(43) Pub. Date: Jul. 3, 2025

(54) PHYSIOLOGICAL STATE CONTROL SYSTEM, PHYSIOLOGICAL STATE CONTROL METHOD, AND NON-TRANSITORY COMPUTER READABLE MEDIUM

(71) Applicant: TOYOTA JIDOSHA KABUSHIKI KAISHA, Toyota-shi (JP)

(72) Inventors: Keiji HAYASHI, Toyota-shi (JP); Hitoshi YAMADA, Toyota-shi (JP); Yuhei YAMAGUCHI, Toyota-shi (JP)

(73) Assignee: TOYOTA JIDOSHA KABUSHIKI KAISHA, Toyota-shi (JP)

(21) Appl. No.: 18/985,407

(22) Filed: Dec. 18, 2024

(30) Foreign Application Priority Data
Dec. 27, 2023 (JP) 2023-221200

(51) Int. Cl. A61H 23/00

(52) U.S. Cl. CPC A61H 23/00; A61H 2201/5005; A61H 2205/04; A61H 2230/105

(57) ABSTRACT

A physiological state control system, a physiological state control method, and an oscillation wave calculation program capable of bringing the autonomic nerve system close to normal while reducing a load on a living body are provided. A physiological state control system according to this embodiment includes: a waveform measurement device configured to acquire waveform information on a living body; a physiological state control apparatus configured to calculate oscillation waves to be imparted to the living body based on the waveform information acquired by the waveform measurement device; and a drive apparatus configured to impart the calculated oscillation waves to at least one of a carotid artery bifurcation or a vertebral artery part of the living body from the outside of the living body.

### CROSS REFERENCE TO RELATED APPLICATIONS

This application is based upon and claims the benefit of priority from Japanese patent application No. 2023-221200, filed on Dec. 27, 2023, the disclosure of which is incorporated herein in its entirety by reference.

### BACKGROUND

The present disclosure relates to a physiological state control system, a physiological state control method, and an oscillation wave calculation program.

Patent Literature 1 discloses a technique for normalizing an autonomic nerve system by sensing a human activity and applying an appropriate nerve stimulus based on this activity.
[Patent Literature 1] Published Japanese Translation of PCT International Publication for Patent Application, No. 2010-508969
[Non-Patent Literature 1] W Zong, T Heldt, G B Moody and R G Mark, “An Open-source Algorithm to Detect Onset of Arterial Blood Pressure Pulses”, Computers in Cardiology 2003; 30:259-262. [online], [searched on Aug. 12, 2021], Internet <https://lcp.mit.edu/pdf/Zong03a.pdf>

### SUMMARY

In the method disclosed in Patent Literature 1, a nerve stimulation signal is applied by an implantable medical device such as a pacemaker, which causes a large load on a living body as this device is implanted inside the body.

The present disclosure has been made in order to solve the aforementioned problem, and provides a physiological state control system, a physiological state control method, and an oscillation wave calculation program capable of bringing the autonomic nerve system close to normal while reducing the load on the living body.

A physiological state control system according to this embodiment includes: a waveform measurement device configured to acquire waveform information of pulse waves of carotid artery bifurcations and vertebral arteries regarding a living body and brain waves of each part measured by the 10-20 method or the like, a physiological state control apparatus configured to calculate oscillation waves to be imparted to the living body based on the waveform information acquired by the waveform measurement device, and a drive apparatus configured to impart the calculated oscillation waves to at least one of the carotid artery bifurcations and vertebral arteries regarding the living body from the outside of the living body. According to this configuration, it is possible to bring the autonomic nerve system close to normal while reducing the load on the living body.

The autonomic nerves work 24 hours a day, 7 days a week to regulate the activities of the circulatory, digestive, and respiratory systems, regardless of a person's will. Because the autonomic nerves respond automatically, they unconsciously regulate functions such as breathing, blood circulation, thermoregulation, digestion, excretion, reproduction, immunity, etc., and are indispensable for maintaining the person's life. When the autonomic nerves become hypertonic, symptoms of dysautonomia such as lethargy, constipation, diarrhea, headache, hot flash, palpitation, and numbness appear. It is appreciated that it is the manifestation of brain functions such as the brainstem to control the above physiological states.

The paths of flow of cerebral blood for supplying energy sources for brain activity are as follows. Blood that has absorbed oxygen in the lungs is sent from the left atrium to the left ventricle, and then the blood is sent from the aortic arch to the rest of the body. The right and left common carotid arteries branched out from the aortic arch each branch out to the internal carotid artery at the carotid artery bifurcation to supply blood to the frontal lobe, the temporal lobe, and the parietal lobe, and the right and left subclavian arteries branch out to the right and left respective vertebral arteries to supply blood to the brainstem, the cerebellum, and the occipital lobe along the cervical vertebra.

By measuring the waveforms in the above parts by the waveform measurement device, information on the blood flow supplied by these arteries can be obtained, and by observing the brain waves of each part of the brain, the state of brain activity can be observed from both sides of the cerebral blood flow and the brain waves.

In the above physiological state control system, the physiological state control apparatus filters the acquired waveform information in at least one frequency band, performs Hilbert transformation on the waveform information filtered in the frequency band, extract a real part and an imaginary part of a complex waveform equation obtained by performing Hilbert transformation on the waveform information, calculating, assuming that the real part is an instantaneous amplitude and the imaginary part is an instantaneous phase, an instantaneous frequency, which is a time differentiation of the instantaneous amplitude and the instantaneous phase in a period in which the living body can be regarded as being in a physiologically steady state, and calculates each distribution. Then the distribution data is distributed like a Gaussian distribution.

In the above physiological state control system, the physiological state control apparatus may include, as the frequency band, at least one of bands of 0.004-0.015 Hz (VLF2), 0.015-0.04 Hz (VLF1), 0.04-0.15 Hz (LF), 0.15-0.4 Hz (HF), 0.4-1.5 Hz (δ1), 1.5-4 Hz (δ2), 4-8 Hz (θ), 8-13 Hz (α), 13-30 Hz (β), and 30-Hz (γ). Further, the frequency band may be further divided into a plurality of segments in δ1, δ2, θ, α, β, and γ bands equal to or higher than 1.5 Hz. According to this configuration, it is possible to acquire the physiological state from brain waves and pulse waves.

In the above physiological state control system, it is observed that, when symptoms of dysautonomia appear, a mean value and a variance value of the amplitude and frequency distributions in at least one of bands of VLF2, VLF1, LF, HF, δ1, δ2, θ, α, β, and γ are deviated from the normal distribution. The physiological state control apparatus calculates an instantaneous amplitude and an instantaneous frequency which change a mean value and a variance value of amplitudes and frequencies when the autonomic nerves are abnormal to the distributions of amplitudes and frequencies when the autonomic nerves are normal, and makes the blood flowing to the brainstem and the brain close to the normal values by applying oscillations or pressure to the drive apparatus, thereby improving dysautonomia. According to this configuration, it is possible to bring the physiological state close to the normal state.

In the above physiological state control system, the physiological state control apparatus may calculate, as the waveform information, the oscillation waves to be imparted to the living body based on at least one of brain waves, pulse waves, or pulse interval waves. Further, whether or not the physiological state is approaching the normal values may be determined by checking the distribution shapes of the instantaneous amplitude and the instantaneous frequency of a brain wave band waveform and an amplitude at which the drive apparatus is driven may be adjusted. According to this configuration, it is possible to bring the physiological state close to the normal state.

A physiological state control method according to this embodiment includes: acquiring waveform information of at least one of brain waves or pulse waves regarding a living body; calculating, based on the acquired waveform information, oscillation waves to be imparted to the living body; and imparting the calculated oscillation waves to at least one of right and left carotid artery bifurcations and right and left vertebral arteries (right and left cervical vertebrae) of the living body from the outside of the living body. According to this configuration, it is possible to bring the autonomic nerve system close to normal while reducing the load on the living body.

In the above physiological state control method, the calculation of the oscillation wave includes: filtering the acquired waveform information in at least one frequency band; performing Hilbert transformation on the waveform information filtered in the frequency band; calculating an instantaneous frequency corresponding to a time differential value of an instantaneous amplitude corresponding to a real part of a complex waveform equation obtained by performing Hilbert transformation on the waveform information and an instantaneous phase corresponding to an imaginary part of the complex waveform equation obtained by performing Hilbert transformation on the waveform information; calculating a distribution of the instantaneous values distributed like a Gaussian distribution; and calculating the oscillation wave in such a way that the calculated Gaussian-like distribution becomes a Gaussian-like distribution in the normal physiological state, storing distribution shapes of an instantaneous amplitude and an instantaneous frequency in the normal physiological state and distribution shapes of an instantaneous amplitude and an instantaneous frequency in a state in which there is an abnormality in the autonomic nerves.

In the above physiological state control method, in the above filtering, at least one of bands of 0.004-0.015 Hz (VLF2), 0.015-0.04 Hz (VLF1), 0.04-0.15 Hz (LF), 0.15-0.4 Hz (HF), 0.4-1.5 Hz (δ1), 1.5-4 Hz (δ2), 4-8 Hz (θ), 8-13 Hz (α), 13-30 Hz (β), and 30-100 Hz (γ) may be included as the frequency band.

In the above physiological state control method, as the waveform information, distributions of an instantaneous amplitude and an instantaneous frequency in each band and each part are measured based on at least one of brain waves, pulse waves, and a pulse interval waveform, a band and an part where the distribution shapes are deviated from those in the normal physiological state are specified, an oscillation waveform having a distribution of the instantaneous amplitude and the instantaneous frequency in the bands in the normal physiological state is input to the drive apparatus of one of the right and left carotid artery bifurcations and the right and left vertebral arteries related to the above band and part, and the blood flow of the above part is made close to that in the normal state, whereby the abnormality of the autonomic nerve system can be improved.

When there are a plurality of bands where the distribution shapes are deviated from those in the normal physiological state at a specific part, the oscillation waveform in the normal physiological state input to the drive apparatus may be the one in which oscillation waveforms in a plurality of bands are superimposed on each other.

According to this embodiment, it is possible to provide a physiological state control system, a physiological state control method, and an oscillation wave calculation program capable of bringing the autonomic nerve system close to normal while reducing a load on a living body.

The above and other objects, features and advantages of the present disclosure will become more fully understood from the detailed description given hereinbelow and the accompanying drawings.

### BRIEF DESCRIPTION OF DRAWINGS

FIG. 1 is a diagram illustrating a configuration of a physiological state control system according to a first embodiment;

FIG. 2 is a block diagram illustrating a physiological state control apparatus according to the first embodiment;

FIG. 3 is a flowchart illustrating a physiological state control method according to the first embodiment;

FIG. 4 is a graph illustrating waveform information on a living body according to the first embodiment, in which the horizontal axis indicates a time and the vertical axis indicates an intensity;

FIG. 5 is a flowchart illustrating a method for calculating oscillation waves performed by the physiological state control apparatus according to the first embodiment;

FIG. 6 is a graph illustrating signal waveforms obtained by performing filtering processing on pulse waves in each frequency band as the waveform information on the living body according to the first embodiment, in which the horizontal axis indicates a time and the vertical axis indicates an intensity;

FIG. 7 is a graph illustrating signal waveforms obtained by performing filtering processing on brain waves in each frequency band as the waveform information on the living body according to the first embodiment, in which the horizontal axis indicates a time and the vertical axis indicates an intensity;

FIG. 8 is a diagram illustrating each frequency band when the waveform information on the living body is filtered according to the first embodiment;

FIG. 9 is a graph illustrating signal waveforms obtained by performing Fourier transformation on the waveform information on the living body according to the first embodiment, in which the horizontal axis indicates a frequency and the vertical axis indicates a spectral power;

FIG. 10 is a diagram showing a complex waveform equation obtained by performing Hilbert transformation on the waveform information on the living body on a complex plane according to the first embodiment;

FIG. 11A is a diagram illustrating a probability density distribution and a Gaussian distribution of instantaneous values according to the first embodiment;

FIG. 11B is a diagram illustrating a probability density distribution and a Gaussian distribution of instantaneous values according to the first embodiment;

FIG. 11C is a diagram illustrating a probability density distribution and a Gaussian distribution of instantaneous values according to the first embodiment;

FIG. 11D is a diagram illustrating a probability density distribution and a Gaussian distribution of instantaneous values according to the first embodiment;

FIG. 12A is a diagram illustrating a probability density distribution of an instantaneous phase difference of HF bands according to the first embodiment;

FIG. 12B is a diagram illustrating a probability density distribution of an instantaneous phase difference of LF bands according to the first embodiment;

FIG. 13A is a diagram illustrating a probability density distribution of a logarithmic amplitude and a frequency in θ bands according to the first embodiment;

FIG. 13B is a diagram illustrating a probability density distribution of a logarithmic amplitude and a frequency in α bands according to the first embodiment;

FIG. 13C is a diagram illustrating a probability density distribution of a logarithmic amplitude and a frequency in θ bands according to the first embodiment;

FIG. 13D is a diagram illustrating a probability density distribution of a logarithmic amplitude and a frequency in α bands according to the first embodiment;

FIG. 14A is a diagram illustrating a probability density distribution of a phase difference between brain waves and pulse waves according to the first embodiment;

FIG. 14B is a diagram illustrating a probability density distribution of a phase difference between brain waves and pulse waves according to the first embodiment;

FIG. 15A is a diagram illustrating a probability density distribution and a Gaussian distribution of an instantaneous frequency in brain waves of the left brain during rest with eyes opened according to the first embodiment;

FIG. 15B is a diagram illustrating a probability density distribution and a Gaussian distribution of an instantaneous frequency in brain waves of the left brain during rest with eyes opened according to the first embodiment;

FIG. 15C is a diagram illustrating a probability density distribution and a Gaussian distribution of an instantaneous frequency in brain waves of the left brain during rest with eyes opened according to the first embodiment;

FIG. 15D is a diagram illustrating a probability density distribution and a Gaussian distribution of an instantaneous frequency in brain waves of the left brain during rest with eyes opened according to the first embodiment;

FIG. 16A is a diagram illustrating a probability density distribution and a Gaussian distribution of an instantaneous frequency in brain waves of the left brain during rest with eyes opened according to the first embodiment;

FIG. 16B is a diagram illustrating a probability density distribution and a Gaussian distribution of an instantaneous frequency in brain waves of the left brain during rest with eyes opened according to the first embodiment;

FIG. 17A is a diagram illustrating a probability density distribution and a Gaussian distribution of an instantaneous frequency in brain waves of the right brain during rest with eyes opened according to the first embodiment;

FIG. 17B is a diagram illustrating a probability density distribution and a Gaussian distribution of an instantaneous frequency in brain waves of the right brain during rest with eyes opened according to the first embodiment;

FIG. 17C is a diagram illustrating a probability density distribution and a Gaussian distribution of an instantaneous frequency in brain waves of the right brain during rest with eyes opened according to the first embodiment;

FIG. 17D is a diagram illustrating a probability density distribution and a Gaussian distribution of an instantaneous frequency in brain waves of the right brain during rest with eyes opened according to the first embodiment;

FIG. 18A is a diagram illustrating a probability density distribution and a Gaussian distribution of an instantaneous frequency in brain waves of the right brain during rest with eyes opened according to the first embodiment;

FIG. 18B is a diagram illustrating a probability density distribution and a Gaussian distribution of an instantaneous frequency in brain waves of the right brain during rest with eyes opened according to the first embodiment;

FIG. 19A is a diagram illustrating a probability density distribution and a Gaussian distribution of an instantaneous frequency in brain waves of the left brain during rest with eyes closed according to the first embodiment;

FIG. 19B is a diagram illustrating a probability density distribution and a Gaussian distribution of an instantaneous frequency in brain waves of the left brain during rest with eyes closed according to the first embodiment;

FIG. 19C is a diagram illustrating a probability density distribution and a Gaussian distribution of an instantaneous frequency in brain waves of the left brain during rest with eyes closed according to the first embodiment;

FIG. 19D is a diagram illustrating a probability density distribution and a Gaussian distribution of an instantaneous frequency in brain waves of the left brain during rest with eyes closed according to the first embodiment;

FIG. 20A is a diagram illustrating a probability density distribution and a Gaussian distribution of an instantaneous frequency in brain waves of the left brain during rest with eyes closed according to the first embodiment;

FIG. 20B is a diagram illustrating a probability density distribution and a Gaussian distribution of an instantaneous frequency in brain waves of the left brain during rest with eyes closed according to the first embodiment;

FIG. 21A is a diagram illustrating a probability density distribution and a Gaussian distribution of an instantaneous frequency in brain waves of the right brain during rest with eyes closed according to the first embodiment;

FIG. 21B is a diagram illustrating a probability density distribution and a Gaussian distribution of an instantaneous frequency in brain waves of the right brain during rest with eyes closed according to the first embodiment;

FIG. 21C is a diagram illustrating a probability density distribution and a Gaussian distribution of an instantaneous frequency in brain waves of the right brain during rest with eyes closed according to the first embodiment;

FIG. 21D is a diagram illustrating a probability density distribution and a Gaussian distribution of an instantaneous frequency in brain waves of the right brain during rest with eyes closed according to the first embodiment;

FIG. 22A is a diagram illustrating a probability density distribution and a Gaussian distribution of an instantaneous frequency in brain waves of the right brain during rest with eyes closed according to the first embodiment;

FIG. 22B is a diagram illustrating a probability density distribution and a Gaussian distribution of an instantaneous frequency in brain waves of the right brain during rest with eyes closed according to the first embodiment;

FIG. 23 is a diagram illustrating a complex transformation expression in each frequency band in a three dimensional manner according to the first embodiment, in which the horizontal axis and the vertical axis on a complex plane indicate Re(φ(t)) and Im(φ(t)) of a complex transformation expression and an axis orthogonal to the complex plane indicates a frequency;

FIG. 24 is a diagram illustrating a complex transformation expression in each frequency band in a three dimensional manner according to the first embodiment, in which the horizontal axis and the vertical axis on a complex plane indicate Re(φ(t)) and Im(φ(t)) of a complex transformation expression and an axis orthogonal to the complex plane indicates a frequency;

FIG. 25 shows a graph illustrating a relationship between a total hemoglobin concentration and a frequency, in which the horizontal axis indicates the frequency and the vertical axis indicates the total hemoglobin concentration;

FIG. 26 is a diagram illustrating definitions of a pulse wave waveform and a pulse interval according to a second embodiment;

FIG. 27 is a diagram illustrating definitions of pulse interval waves according to the second embodiment;

FIG. 28 is a diagram illustrating time-series data of a pulse wave instantaneous amplitude for each band in a normal physiological state; and

FIG. 29 is a diagram illustrating time-series data of a pulse wave instantaneous frequency for each band in the normal physiological state.

### DESCRIPTION OF EMBODIMENTS

Embodiments of the present disclosure will now be described. However, the claimed disclosure is not limited to the following embodiments. Moreover, not all of the configurations described in the embodiments are essential as means for solving the problem. For the sake of clarity, the following descriptions and drawings are omitted and simplified as appropriate. In each drawing, the same elements have the same reference signs, and repeated descriptions have been omitted as appropriate.

### First Embodiment

A physiological state control system according to first embodiment will be described. The physiological state control system according to this embodiment acquires, for example, waveform information on a living body, such as a brain wave waveform and a pulse wave waveform, and calculates, based on the acquired waveform information, oscillation waves to be imparted to the living body. The pulse wave waveform may be a photoelectric plethysmographic waveform or a piezoelectric waveform in which a pulse pressure is detected. The pulse wave waveform may also be a Doppler effect processing waveform reflected on an arterial wall when ultrasound is used or time-series data of an arterial diameter calculated from a moving image of a cross section of an artery by ultrasound tomography. Brain waves in a plurality of parts of the head are simultaneously measured by the 10-20 method or the like. The living body is, for example, a person who is a subject. For example, the physiological state control system acquires waveform information of a normal physiological state regarding the living body, calculates feature amounts of predetermined oscillation waves in which a central frequency and an amplitude are set, and records feature amounts of the normal physiological state (a mean value of the amplitude and the frequency and a variance value of the amplitude and frequency in each frequency band). Then the physiological state control system calculates feature amounts of the oscillation waves in the current physiological state, and imparts oscillation waves so as to compensate for feature amounts that are different from the feature amounts of the oscillation waves in the normal physiological state (the mean value of the amplitude and the frequency and the variance value of the amplitude and the frequency in each frequency band) from the outside of the living body, thereby controlling the physiological state of the living body. In the following, <Configuration of Physiological State Control System> will be described first. After that, <Physiological State Control Method> which uses the physiological state control system will be described.

### <Configuration of Physiological State Control System>

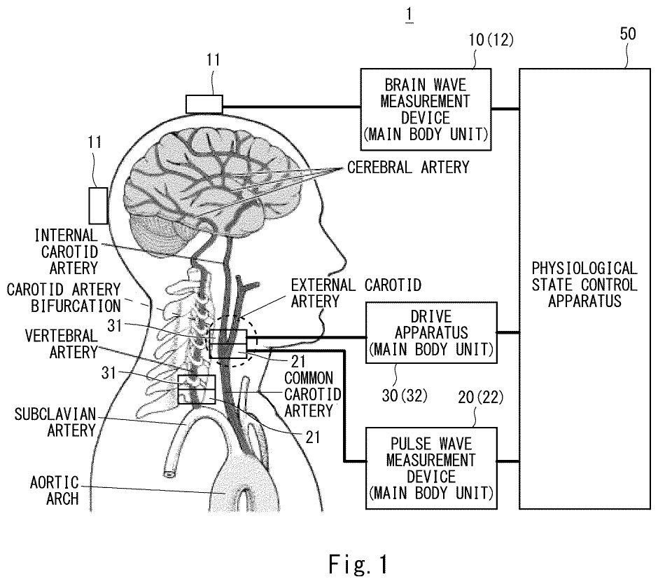

FIG. 1 is a diagram illustrating a configuration of a physiological state control system according to the first embodiment. As shown in FIG. 1, a physiological state control system 1 includes a brain wave measurement device 10, a pulse wave measurement device 20, a drive apparatus 30, and a physiological state control apparatus 50. A device such as the brain wave measurement device 10 and the pulse wave measurement device 20 which acquires waveform information on a living body is called a waveform measurement device.

The brain wave measurement device 10 acquires, as waveform information on a living body, brain waves of the living body. The brain wave measurement device 10 includes a sensor 11 and a main body unit 12. The sensor 11 is placed in each of a plurality of parts by the 10-20 method or the like, and the main body unit 12 performs measurement simultaneously. The sensor 11 is attached, for example, onto a scalp of a person's head, and senses information on the brain waves of the person from the outside of the living body. The information on the brain waves of the person is, for example, a voltage. The sensor 11 may sense, as information on the brain waves of the person, other than the voltage, a current, a magnetic field or the like. The sensor 11 is attached to the living body in a non-invasive manner.

The sensor 11 outputs information on the sensed brain waves to the main body unit 12 of the brain wave measurement device 10. The main body unit 12 of the brain wave measurement device 10 measures a temporal change in a voltage or the like output from the sensor 11. The sensor 11 is connected to the main body unit 12 by a wired or wireless communication line. Further, the main body unit 12 is connected to the physiological state control apparatus 50 by a wired or wireless communication line. The main body unit 12 outputs the measured brain waves to the physiological state control apparatus 50.

The pulse wave measurement device 20 measures pulse waves of a living body as waveform information on the living body. The pulse waves are waveform information on the living body formed by a pulse interval, a blood output, and physical characteristics of blood vessels. The pulse wave measurement device 20 includes a sensor 21 and a main body unit 22. The pulse wave measurement device 20 may be either a photoelectric device or a piezoelectric device. When the photoelectric type device is used, a near-infrared light in which the light wavelength is from 800 to 1000 nm may be used. The sensor 21 is attached, for example, onto a skin on the neck (carotid artery bifurcations) or cervical vertebrae (vertebral arteries) of the person, and measures information on the pulse waves flowing into the brain of the person from the outside of the living body. Specifically, the sensor 21 may be disposed in at least one of a part near the right and left carotid artery bifurcations or a part near the right and left vertebral arteries (cervical vertebrae). The information on the pulse waves of the person is, for example, a pulse pressure. The sensor 21 may sense, besides the pulse pressure, an amount of the blood flow as the information on the pulse waves of a person. The sensor 21 is attached to a living body in a non-invasive manner.

The sensor 21 outputs the sensed information on the pulse waves to the main body unit 22 of the pulse wave measurement device 20. The main body unit 22 of the pulse wave measurement device 20 measures a temporal change of a pulse pressure or the like output from the sensor 21. The sensor 21 is connected to the main body unit 22 by a wired or wireless communication line. Further, the main body unit 22 is connected to the physiological state control apparatus 50 by a wired or wireless communication line. The main body unit 22 outputs the measured pulse waves to the physiological state control apparatus 50.

The drive apparatus 30 imparts the oscillation waves calculated by the physiological state control apparatus 50 to at least one of parts near right and left internal carotid arteries (each artery flows into the brain from the carotid artery bifurcation) and parts near right and left vertebral arteries (each artery flows into the brainstem along the cervical vertebra) that supply blood from outside the living body to the brain. The drive apparatus 30 includes an oscillator 31 and a main body unit 32. The oscillator 31 is attached, for example, onto the skin on the neck, the collarbone, or the cervical vertebrae of the person (the vertebral arteries passes along the cervical vertebrae) and imparts the oscillation waves to the person. Specifically, the oscillator 31 may be disposed in at least one of a part near the right and left carotid artery bifurcations and vertebral arteries (cervical vertebrae). In this case, the drive apparatus 30 imparts the calculated oscillation waves to at least one of the right and left carotid artery bifurcations and the right and left vertebral arteries (cervical vertebrae) of the living body from the outside of the living body. This changes the pulse waves of the living body. One or more oscillators 31 may be mounted on the living body. The oscillator 31 is attached to the living body in a non-invasive manner.

The oscillator 31 is connected to the main body unit 32 by a wired or wireless communication line. Further, the main body unit 32 is connected to the drive apparatus 30 by a wired or wireless communication line. The main body unit 32 receives information on the oscillation waves calculated by the physiological state control apparatus 50 from the physiological state control apparatus 50. The information on the oscillation waves to be input to the drive apparatus is, for example, a mean value of an amplitude and a frequency in the oscillation waves, and a variance value of the amplitude and the frequency in the oscillation waves. In the oscillation waves, an amplitude and a frequency of at least one of bands of VLF2, VLF1, LF, HF, δ1, δ2, θ, α, β, and γ are each distributed like a Gaussian distribution. The main body unit 32 amplifies the information on the oscillation waves received from the physiological state control apparatus 50 to drive the oscillator 31. The oscillator 31, which is able to add an oscillation in which desired waveforms of a plurality of bands are superimposed on one another to the living body, imparts the oscillation waves from the outside of the living body. In this manner, the drive apparatus 30 imparts the oscillation waves to at least one of the right and left carotid artery bifurcations and the right and left vertebral arteries (cervical vertebrae) of the living body from the outside of the living body.

The physiological state control apparatus 50 stores feature amounts of the oscillation waves of the living body in a normal physiological state based on the waveform information acquired by the waveform measurement device. The feature amounts indicate a mean value of an amplitude and a frequency in the oscillation waves and a variance value of the amplitude and frequency in the oscillation waves. The oscillation waves are waveforms obtained through a band-pass filter in the bands of VLF2, VLF1, LF, HF, δ1, δ2, θ, α, β, and γ. When the probability density distribution of each of the amplitude and the frequency is calculated in a period in which the living body can be regarded as being in a physiologically steady state, they are each distributed like a Gaussian distribution. When symptoms of dysautonomia appear, similar feature amounts are calculated to detect the difference from the feature amounts in the normal physiological state, and an oscillation waveform so as to compensate for this difference is generated. By inputting this oscillation waveform into the drive apparatus and driving the oscillator 31, the state of the blood flow flowing into the brain can be brought close to the normal state and the symptoms of dysautonomia can be improved.

Specifically, it is assumed that, due to dysautonomia, the mean of the amplitude distribution in a certain band of the pulse waves in the right internal carotid artery has been changed from M0 to M1, the variance of the amplitude distribution therein from S0 to S1, the mean of the frequency distribution therein has been changed from m0 to m1, and the variance therein from s0 to s1. At this time, if parts where there are modulations in the distributions of amplitudes and frequencies in that band in the brain waves has been found in the right temporal lobe, parietal lobe, and frontal lobe, the oscillator 31 is applied to the right internal carotid artery while monitoring the degree of recovery of the modulations, and an oscillation in which the mean of the distribution of the amplitude is made to approach from M1 to M0, the variance thereof is made to approach from S1 to S0, the mean of the frequency is made to approach from m1 to m0, and the variance thereof is made to approach from s1 to s0 is applied. Accordingly, the symptoms of dysautonomia can be improved. The reason for not abruptly giving the oscillation distribution in the normal physiological state is to avoid applying stress to the living body. The same holds true for a case where the system of the artery flowing into the brain is different or a band in which a modulation is found is different. Further, in a case where modulations have been found simultaneously in a plurality of bands as well, an oscillation distribution waveform that improves the bands in which modulations are found may be superimposed on one another and the obtained waveform may be imparted to a desired oscillator.

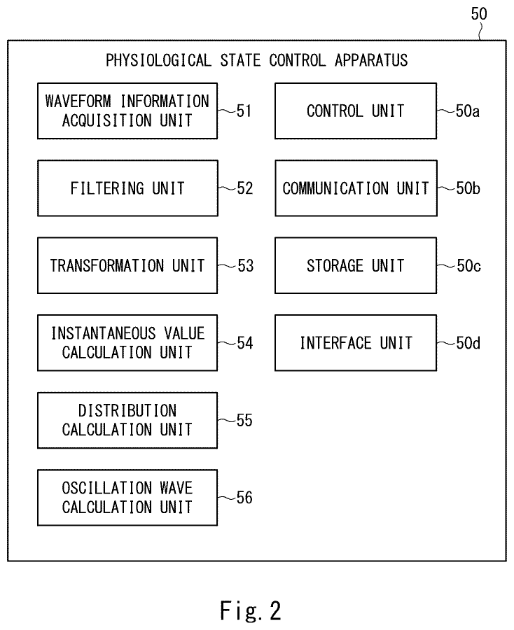

FIG. 2 is a block diagram illustrating the physiological state control apparatus 50 according to the first embodiment. As shown in FIG. 2, the physiological state control apparatus 50 includes a control unit 50a, a communication unit 50b, a storage unit 50c, an interface unit 50d, a waveform information acquisition unit 51, a filtering unit 52, a transformation unit 53, an instantaneous value calculation unit 54, a distribution calculation unit 55, and an oscillation wave calculation unit 56. The control unit 50a, the communication unit 50b, the storage unit 50c, the interface unit 50d, the waveform information acquisition unit 51, the filtering unit 52, the transformation unit 53, the instantaneous value calculation unit 54, the distribution calculation unit 55, and the oscillation wave calculation unit 56 respectively include functions as control means, communication means, storing means, interface means, waveform information acquisition means, filtering means, transformation means, instantaneous value calculation means, distribution calculation means, and oscillation wave calculation means.

The physiological state control apparatus 50 is an information processing apparatus including a computer. The control unit 50a includes, for example, a processor such as a Central Processing Unit (CPU), a Micro Processing Unit (MPU), an Electronic Control Unit (ECU), a Field-Programmable Gate Array (FPGA), or an Application Specific Integrated Circuit (ASIC). The control unit 50a includes a function as an arithmetic apparatus that performs control processing, arithmetic processing, and so on. Further, the control unit 50a controls the operations of the respective components of the communication unit 50b, the storage unit 50c, the interface unit 50d, the waveform information acquisition unit 51, the filtering unit 52, the transformation unit 53, the instantaneous value calculation unit 54, the distribution calculation unit 55, the oscillation wave calculation unit 56, and so on.

Each of the components of the physiological state control apparatus 50 can be implemented, for example, by executing a program through control performed by the control unit 50a. More specifically, each of the components may be implemented by the control unit 50a executing the program stored in the storage unit 50c. Further, by recording a necessary program in any non-volatile storage medium and installing the program as necessary, each of the components may be implemented. Further, each of the components is not limited to be implemented by software by a program and may be implemented by, for example, any combination of hardware, firmware, and software.

The communication unit 50b receives the waveform information measured by a waveform measurement device such as the brain wave measurement device 10 and the pulse wave measurement device 20 from the waveform measurement device. The waveform information is, for example, time-series data of brain waves and pulse waves. The communication unit 50b transmits the information on the oscillation waves calculated by the oscillation wave calculation unit 56 to the drive apparatus 30.

The storage unit 50c may include, for example, a storage apparatus such as a memory or a hard disk. The storage apparatus is, for example, a Read Only Memory (ROM) or a Random Access Memory (RAM). The storage unit 50c includes a function for storing a control program, an arithmetic program and the like executed by the control unit 50a. Further, the storage unit 50c includes a function for temporarily storing process data and so on.

The storage unit 50c may store waveform information such as brain waves and pulse waves received by the communication unit 50b. The storage unit 50c may store the oscillation waves calculated by the oscillation wave calculation unit 56. The storage unit 50c may store various kinds of parameters used by the oscillation wave calculation unit 56 to calculate the oscillation waves.

The interface unit 50d is, for example, a user interface. The interface unit 50d includes an input apparatus such as a keyboard, a touch panel, or a mouse, and an output apparatus such as a display or a speaker. The interface unit 50d accepts an operation of inputting data performed by a user (an operator or the like) and outputs information to the user.

The waveform information acquisition unit 51 acquires waveform information that the communication unit 50b has acquired from the waveform measurement device. The filtering unit 52 filters the acquired waveform information in at least one frequency band. The transformation unit 53 performs transformation for transforming the waveform information filtered in the frequency band into a complex number. The transformation for transforming the waveform information into a complex number is, for example, Hilbert transformation. The instantaneous value calculation unit 54 calculates an instantaneous value including at least one of an instantaneous amplitude, which is an amplitude term corresponding to a real part of the complex waveform obtained by converting the waveform information on pulse waves and brain waves into a complex number, an instantaneous frequency, which is a time differential value of a phase term corresponding to an imaginary part thereof, and an instantaneous phase difference corresponding to a difference in the phase term between the brain waves and the pulse waves. The distribution calculation unit 55 calculates a probability density distribution of the instantaneous value. Further, the distribution calculation unit 55 calculates a Gaussian distribution that is fit to the probability density distribution. The oscillation wave calculation unit 56 calculates information on oscillation waves in such a way that the calculated Gaussian distribution becomes a predetermined Gaussian distribution. The oscillation wave calculation unit 56 outputs the information on the calculated oscillation waves to the communication unit 50b. The information on the oscillation waves includes an amplitude and a central frequency. The communication unit 50b outputs the information on the oscillation waves to the drive apparatus 30 and drives the oscillator 31.

### <Physiological State Control Method>

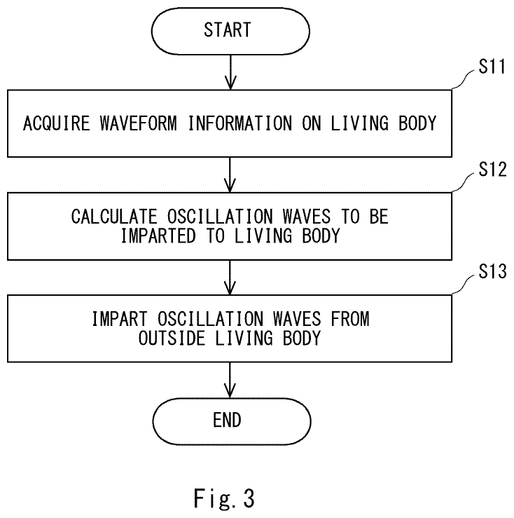

Next, a physiological state control method will be described. FIG. 3 is a flowchart illustrating the physiological state control method according to the first embodiment. As shown in FIG. 3, the physiological state control method includes a step of acquiring waveform information on a living body (Step S11), a step of calculating oscillation waves to be imparted to the living body (Step S12), and a step of imparting the oscillation waves from the outside of the living body (Step S13).

As shown in Step S11 in FIG. 3, first, waveform information on the living body is acquired. For example, a waveform measurement device such as the brain wave measurement device 10 and the pulse wave measurement device 20 acquires brain waves, pulse waves and so on as waveform information on the living body.

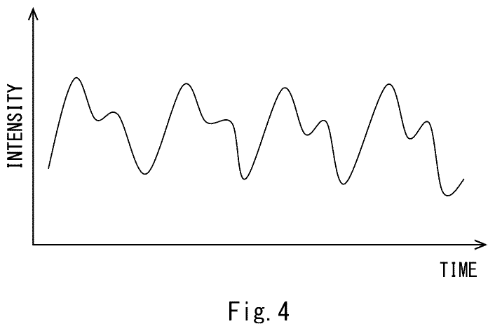

FIG. 4 is a graph illustrating waveform information on a living body according to the first embodiment, in which the horizontal axis indicates a time and the vertical axis indicates an intensity. FIG. 4 shows time-series data of the pulse waves as an example of the waveform information on the living body. Note that the waveform information on the living body is not limited to time-series data of pulse waves and may be time-series data of brain waves or time-series data of a pulse interval (resampled in a predetermined frequency). The pulse wave measurement device 20 acquires, for example, time-series data of the pulse waves via the sensor 21 arranged in the right and left carotid artery bifurcations or the right and left vertebral arteries of the subject. The pulse wave measurement device 20 measures, for example, pulse waves of the subject who is in the steady state for several minutes to several tens of minutes. The sampling frequency is, for example, 500 Hz. Note that the sampling frequency may be any frequency from 10 to 1000 Hz. The pulse wave data thus obtained has a time-series waveform as shown in FIG. 4. The waveform measurement device outputs the acquired waveform information to the physiological state control apparatus 50.

Next, as shown in Step S12, oscillation waves to be imparted to the living body are calculated. The physiological state control apparatus 50 calculates oscillation waves to be imparted to the living body based on the acquired waveform information. A method for calculating the oscillation waves will be described later. The physiological state control apparatus 50 outputs information on the calculated oscillation waves to the drive apparatus 30.

Next, as shown in Step S13, the calculated oscillation waves are imparted from the outside of the living body. For example, the drive apparatus 30 causes the oscillator 31 to generate oscillation waves based on the information on the oscillation waves output from the physiological state control apparatus 50. Accordingly, the oscillator 31 imparts the calculated oscillation waves to, for example, at least one of the left-side left carotid artery bifurcation, the right-side right carotid artery bifurcation, the left-side vertebral artery, or the right-side vertebral artery of the living body from the outside of the living body.

### <Method for Calculating Oscillation Waves>

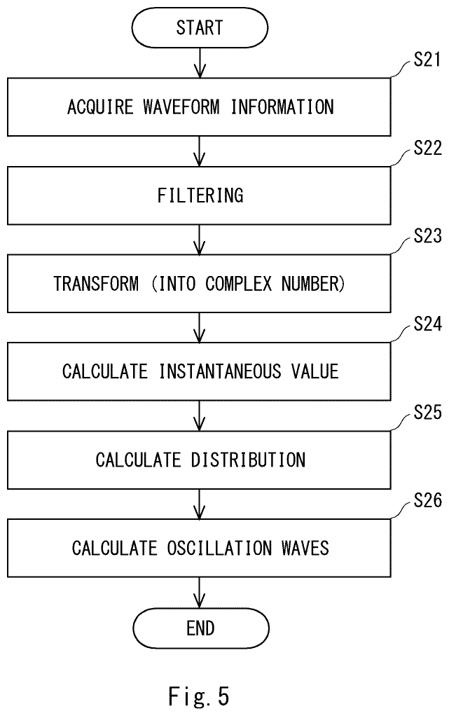

Next, a method for calculating the oscillation waves shown in Step S12 described above will be described. FIG. 5 is a flowchart illustrating a method for calculating the oscillation waves performed by the physiological state control apparatus 50 according to the first embodiment. As shown in FIG. 5, the method for calculating the oscillation waves includes a waveform information acquisition step of acquiring waveform information on a living body (Step S21), a filtering step of filtering the acquired waveform information in at least one frequency band (Step S22), a transformation step of transforming the waveform information filtered in the frequency band into a complex number (Step S23), an instantaneous value calculation step of calculating an instantaneous value including at least one of an instantaneous amplitude, an instantaneous frequency, or an instantaneous phase (Step S24), a distribution calculation step of calculating a distribution of instantaneous values (Step S25), and an oscillation wave calculation step (Step S26) of calculating oscillation waves to be imparted to the living body in such a way that the calculated distribution becomes a predetermined distribution. The above Steps S21 to S25 are performed in a normal physiological state, and numerical values obtained in these steps are stored in the storage unit 50c. Hereinafter, each step will be described.

### <Waveform Information Acquisition Step>

The waveform information acquisition unit 51 of the physiological state control apparatus 50 acquires waveform information that the communication unit 50b has received from a waveform measurement device such as the brain wave measurement device 10, the pulse wave measurement device 20, and so on. The waveform information acquisition unit 51 acquires, for example, a brain wave waveform and a pulse wave waveform from time-series data of the brain waves and the pulse waves.

### <Filtering Step>

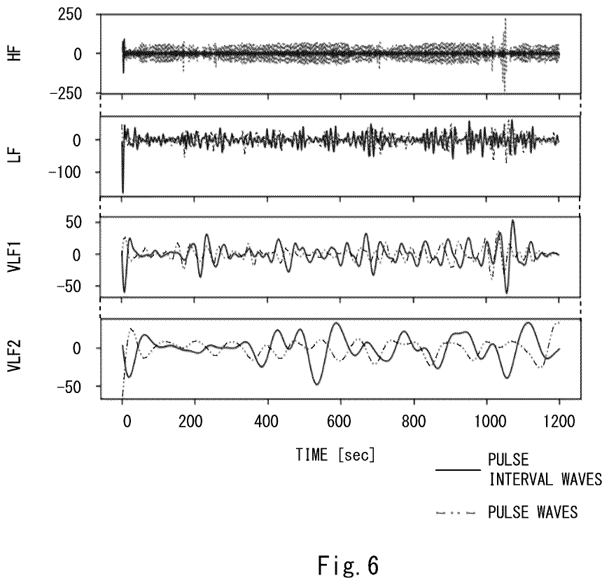

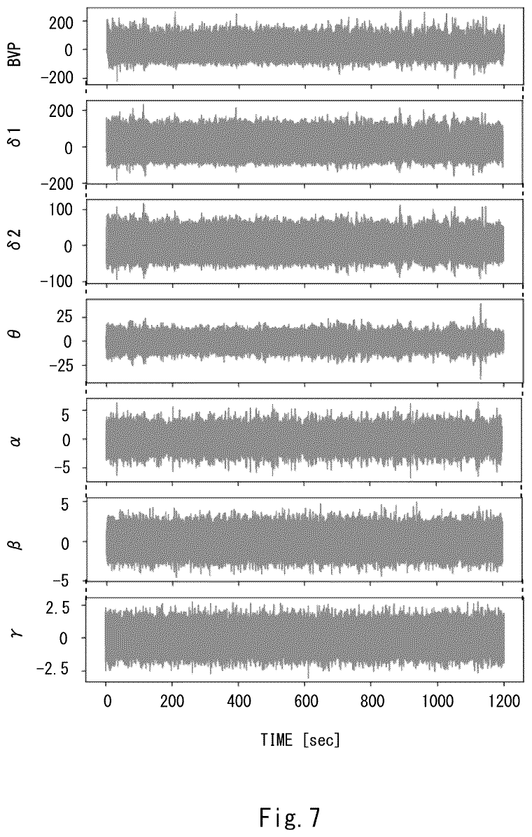

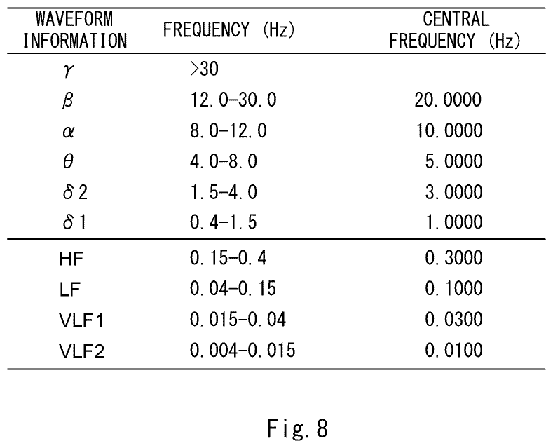

FIG. 6 is a graph illustrating signal waveforms obtained by performing filtering processing on pulse waves in each frequency band as waveform information on a living body according to the first embodiment, in which the horizontal axis indicates a time and the vertical axis indicates an intensity. FIG. 6 show, as each frequency band, HF, LF, VLF1, and VLF2. FIG. 6 also shows pulse interval waveforms that will be described later. FIG. 7 is a graph illustrating signal waveforms obtained by performing filtering processing on brain waves in each frequency band as waveform information on a living body according to the first embodiment, in which the horizontal axis indicates a time and the vertical axis indicates an intensity. FIG. 7 shows band waveforms of brain waves (δ1, δ2, θ, α, β, γ) as frequency bands shorter than the heartbeat interval. FIG. 7 also shows BVP. FIG. 8 is a diagram illustrating each frequency band when the waveform information on a living body is filtered according to the first embodiment. FIG. 8 also shows approximate central frequencies in some frequency bands.

As shown in FIGS. 6 and 7, the filtering unit 52 of the physiological state control apparatus 50 filters the acquired waveform information in at least one frequency band. Specifically, the filtering unit 52 of the physiological state control apparatus 50 filters the waveform information acquired by the waveform information acquisition unit 51 for each frequency band.

As shown in FIGS. 6-8, as the frequency bands to be filtered, for example, VLF2 (0.004-0.015 Hz), VLF1 (0.015-0.04 Hz), LF (0.04-0.15 Hz), HF (0.15-0.4 Hz), δ1 (0.4-1.5 Hz), δ2 (1.5-4 Hz), θ (4-8 Hz), α (8-13 Hz), β (13-30 Hz), γ (30-100 Hz), and the like are selected. Here, VLF1 and VLF2 are frequency bands related to the functions of autonomic nerves, such as thermoregulation, digestion, excretion, reproduction, immunity, etc., HF is a respiratory fluctuation band, and LF is a blood pressure fluctuation band. Signals in frequency bands higher than those, which are put on pulse waves flowing into the brain from the internal carotid artery of the carotid artery bifurcation and the vertebral artery branching from the subclavian artery, include the bands of δ waves (0.4-4.0 Hz), θ waves (4.0-8.0 Hz), α waves (8.0-13.0 Hz), β waves (13.0-30.0), and γ waves (30-100 Hz) in the brain waves.

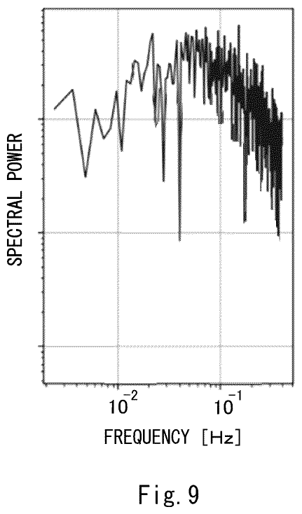

FIG. 9 is a graph illustrating signal waveforms obtained by performing Fourier transformation on the waveform information measured by a commercially available pulse wave meter, in which the horizontal axis indicates a frequency and the vertical axis indicates a spectral power. Since a high-pass filter is provided at about 0.1 Hz, signals at frequencies equal to or below 0.1 Hz are lost. Further, since a green light is irradiated to blood vessels and a reflected light amount is used as a pulse wave signal, only information on capillaries on the surface of the skin can be obtained. Therefore, this signal waveform cannot be employed in this embodiment. For use in this embodiment, and it is required to observe pulse wave signals that has a frequency bandwidth of at least 0.001-100 Hz and is obtained from an artery at a depth of 2 to 3 cm from the skin with near-infrared light (wavelength: 800-1000 nm). Further, in order to observe pulse waves of the vertebral artery that covers a wide frequency band of the living body and is located deeper than the carotid artery, it is required to observe pulse wave signals that has a frequency bandwidth of at least 0.0001-400 Hz and is obtained from an artery at a depth of 2 to 5 cm from the skin with near-infrared light (wavelength: 800-1600 nm).

While brain wave waveforms are not shown in the drawings, filtering processing may be performed in frequency bands similar to those of the pulse wave waveforms.

### <Transformation Step>

Next, the physiological state control apparatus 50 transforms the waveform information filtered in each frequency band into a complex number by Hilbert transformation. Specifically, the transformation unit 53 of the physiological state control apparatus 50 transforms the filtered waveform information such as time-series data φₖ(t) of the filtered waveform in each frequency band as shown in FIGS. 6 and 7 into a complex waveform equation as shown in the following Equation (1) by Hilbert transformation.

$${φ}_{k} ( t ) = exp ( {a}_{k} ( t ) + i {ψ}_{k} ( t ) ) \qquad ( 1 )$$

Here, aₖ(t) is an instantaneous value of a logarithmic amplitude of a k frequency band waveform and ψₖ(t) is an instantaneous value of a phase of the k frequency band waveform.

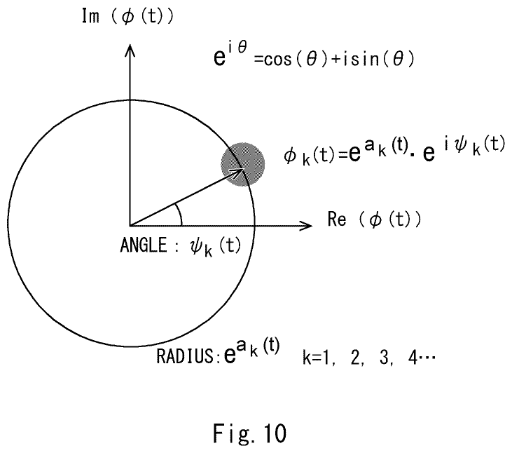

FIG. 10 is a diagram showing a complex waveform equation obtained by performing Hilbert transformation on the waveform information on the living body on a complex plane according to the first embodiment. As shown in FIG. 10, the waveform information (oscillation) indicated by a complex waveform equation can be expressed on a complex plane. When the horizontal axis on the complex plane indicates Re(ln φ(t)) and the vertical axis indicates Im(ln φ(t)), aₖ(t) indicates a radius centered on the origin and ωₖ(t) indicates an angle with the horizontal axis. Here, k=1, 2, 3, 4 . . . indicates each frequency band.

The Hilbert transformation is performed on each frequency band waveform of a pulse wave waveform and a brain wave waveform.

### <Instantaneous Value Calculation Step>

Next, the instantaneous value calculation unit 54 of the physiological state control apparatus 50 calculates an instantaneous value. The instantaneous value includes at least one of an instantaneous logarithmic amplitude, an instantaneous frequency, which is a time differential value of an instantaneous phase, and an instantaneous phase difference. The instantaneous phase difference is a difference in the instantaneous phase between pulse waves and brain waves or a difference in the instantaneous phase between pulse waves and a heartbeat interval. The instantaneous logarithmic amplitude corresponds to a logarithmic real part of a complex waveform equation obtained by performing Hilbert transformation on the waveform information. The instantaneous frequency corresponds to a time differential value of a logarithmic imaginary part of the complex waveform equation. The instantaneous phase difference corresponds to a difference in the imaginary term of the logarithm of the complex transformation expression between brain waves and pulse waves. Specifically, the instantaneous logarithmic amplitude, the instantaneous frequency, and the instantaneous phase difference will be defined by the following Equations (2), (3), and (4).

By performing the processing of Equation (1) on pulse wave data, an instantaneous logarithmic amplitude and an instantaneous frequency in Equations (2) and (3) can be obtained.

$${a}_{k} ( t ) \qquad ( 2 )$$

$${ω}_{k} ( t ) = d {Ψ}_{k} ( t ) / dt \qquad ( 3 )$$

By performing the processing of Equation (1) on brain wave data, an instantaneous logarithmic amplitude and an instantaneous frequency in Equations (4) and (5) can be obtained.

$${A}_{k} ( t ) \qquad ( 4 )$$

$${Ω}_{k} ( t ) = d {Ψ}_{k} ( t ) / dt$$

An instantaneous phase difference in the time-series data of the brain waves and the pulse waves can be expressed as follows.

$${θ}_{k} ( t ) = {Ψ}_{k} ( t ) - {Ψ}_{k} ( t ) \qquad ( 6 )$$

This indicates a phase relationship of the blood flow supplied to the brain that is consumed in each center in the brain, and corresponds to an important feature amount to know the physiological state of each center.

When the distribution of θₖ(t) follows a random process, it can be approximated by a Von Mises distribution shown in Equation (7). Here, Iⱼ(κ) is the modified Bessel function of the first kind with order j.

$$f ( θ ) = exp ( κ cos ( θ - μ ) ) / 2 π {I}_{0} ( κ ) \qquad ( 7 )$$

$${I}_{j} ( κ ) = {( κ / 2 )}^{j} \sum {( {κ}^{2} / 4 )}^{i} / i ! Γ ( j + i + 1 ) \qquad ( 8 )$$

In the above manner, the instantaneous value calculation unit 54 calculates an instantaneous value including at least one of the instantaneous logarithmic amplitudes aₖ(t) and Aₖ(t) corresponding to the real part of the exponential part of the complex waveform equation obtained by performing Hilbert transformation on the waveform information, instantaneous frequencies ωₖ(t) and Ωₖ(t) corresponding to the time differential value of the imaginary part thereof, and an instantaneous phase difference θₖ(t) corresponding to a phase difference in the band waveform between the brain waves and the pulse waves.

### <Distribution Calculation>

Next, the distribution calculation unit 55 of the physiological state control apparatus 50 calculates a distribution of instantaneous values of pulse waves and brain waves. Specifically, the distribution calculation unit 55 calculates a probability density distribution of the instantaneous logarithmic amplitudes aₖ(t) and Aₖ(t), the instantaneous frequencies ωₖ(t) and Ωₖ(t), and the instantaneous phase difference θₖ(t) in a desired time interval. Further, the distribution calculation unit 55 acquires distribution shapes of the instantaneous logarithmic amplitudes aₖ(t) and Aₖ(t), the instantaneous frequencies ωₖ(t) and Ωₖ(t), and the instantaneous phase difference θₖ(t) fit to a monomodal distribution, for example, a Gaussian probability density distribution, at a time interval in which the living body can be regarded as being in a desired physiological steady state in a normal physiological state and in a state in which there are symptoms of dysautonomia, and stores the obtained distribution shapes in the storage unit 50c. Since the value of the instantaneous phase difference is limited to be in a range of −π to +π, a Von Mises distribution is, for example, applied.

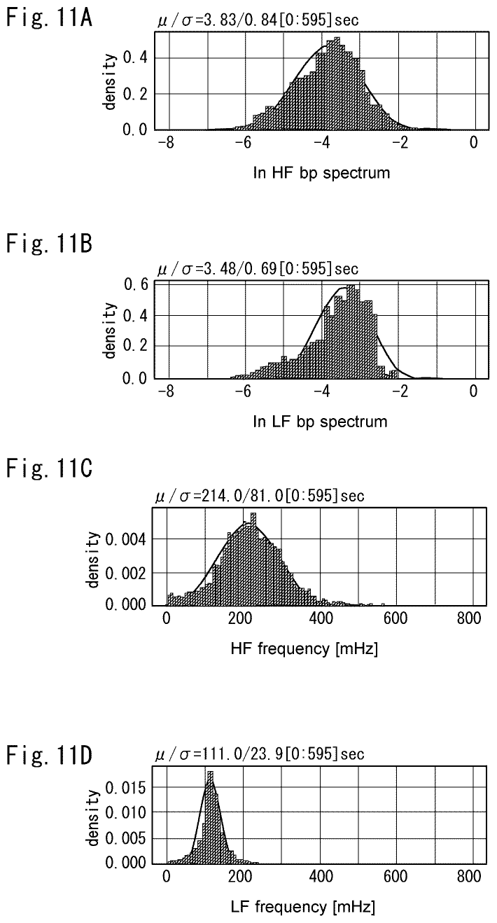

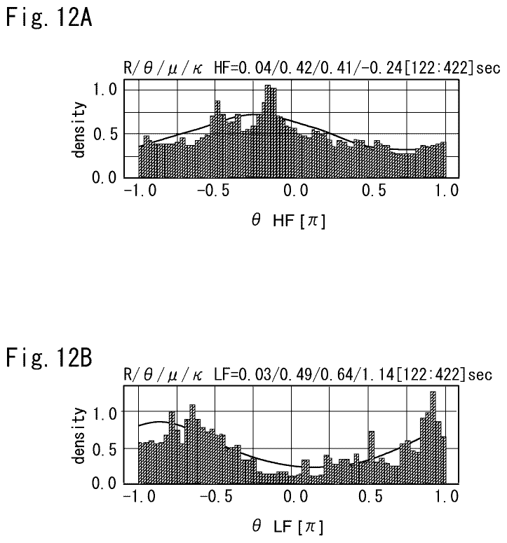

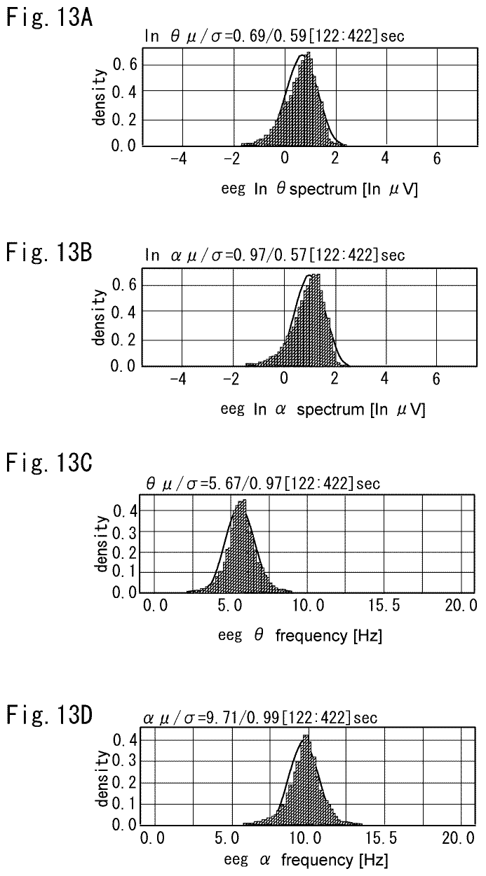

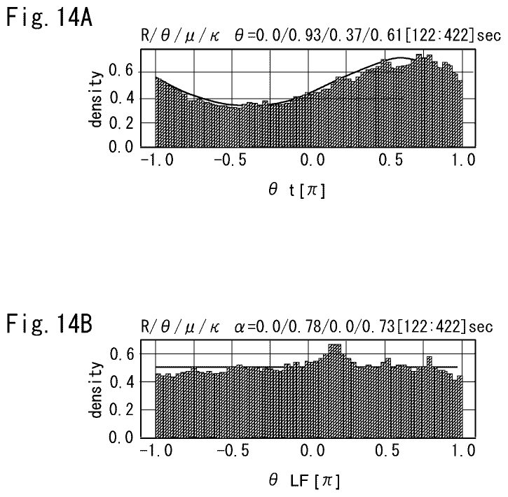

FIG. 11 is a diagram illustrating a probability density distribution and a Gaussian distribution of instantaneous values according to the first embodiment. Each bar graph indicates the probability density distribution and each solid line indicates the Gaussian distribution. FIG. 11A shows a probability density distribution and a Gaussian distribution of an instantaneous logarithmic amplitude of HF and FIG. 11B shows a probability density distribution and a Gaussian distribution of an instantaneous logarithmic amplitude of LF. FIG. 11C shows a probability density distribution and a Gaussian distribution of an instantaneous logarithmic frequency of HF and FIG. 11D shows a probability density distribution and a Gaussian distribution of an instantaneous frequency of LF. A mean value and a variance value of each Gaussian distribution are also shown. In the following drawings of Gaussian distributions as well, mean values and variance values are shown. Each drawing shows a state in which the logarithmic amplitude and the frequency of one oscillator are distributed like a Gaussian distribution for each band. FIGS. 12A-12B show probability density distributions of instantaneous phase differences in HF and LF bands according to the first embodiment, in which solid lines indicate a Von Misese distribution. FIGS. 13A-13D and FIGS. 14A-14B show probability density distributions of logarithmic amplitudes and frequencies in θ and α bands and probability density distributions of phase differences between brain waves and pulse waves according to the first embodiment, in which solid lines in the drawings showing the logarithmic amplitudes and the frequencies are a Gaussian distribution, and solid lines in the drawings showing the phase differences are a Von Misese distribution. Although not shown in the drawings, in frequencies higher than 12 Hz (for example, β and γ bands) as well, the logarithmic amplitudes and the frequencies of each signal waveform are each distributed like a Gaussian distribution.

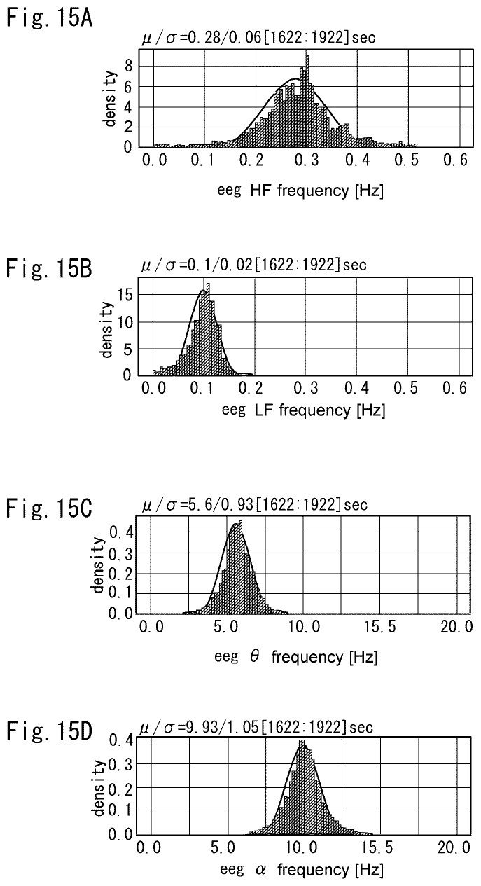

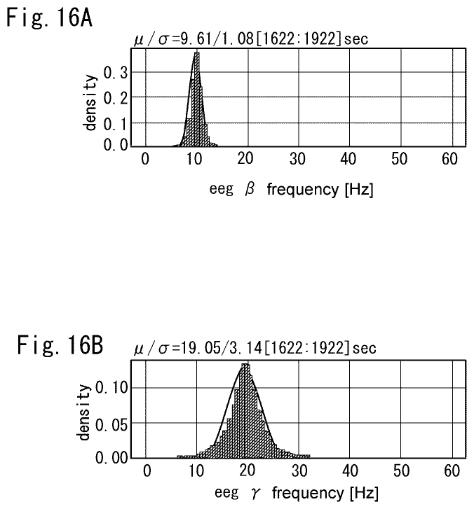

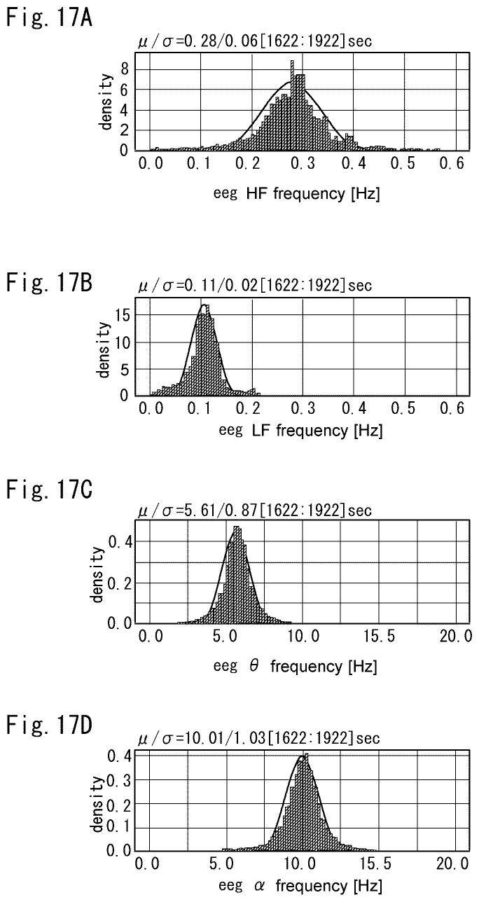

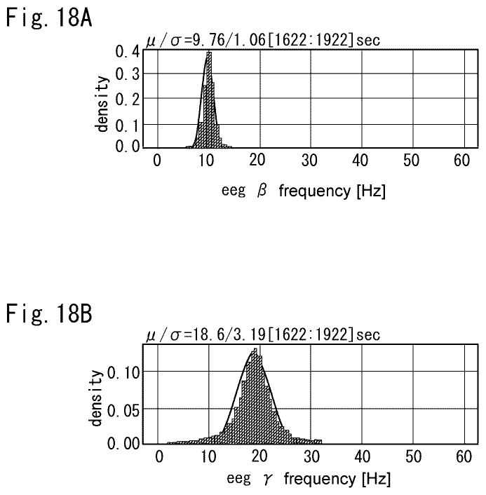

FIGS. 15A-15D and 16A-16B are diagrams illustrating probability density distributions and Gaussian distributions of instantaneous frequencies in bran waves (frontal lobe) of the left brain during rest with eyes opened according to the first embodiment. FIG. 15A shows the HF, FIG. 15B shows the LF, FIG. 15C shows the θ waves, and FIG. 15D shows the α waves. FIG. 16A shows the β waves and FIG. 16B shows the γ waves. FIGS. 17A-17D and FIGS. 18A-18B are diagrams illustrating probability density distributions and Gaussian distributions of instantaneous frequencies in brain waves of the right brain during rest with eyes opened according to the first embodiment. FIG. 17A shows the HF, FIG. 17B shows the LF, FIG. 17C shows the θ waves, and FIG. 17D shows the α waves. FIG. 18A shows the β waves and FIG. 18B shows the γ waves.

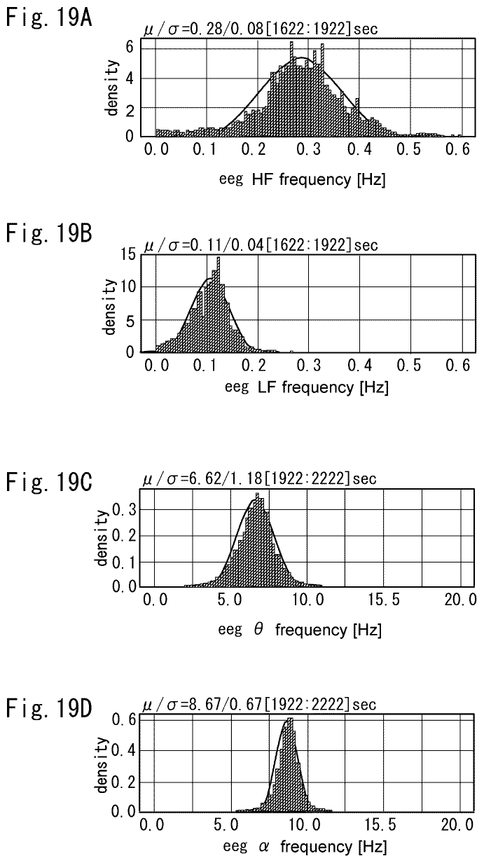

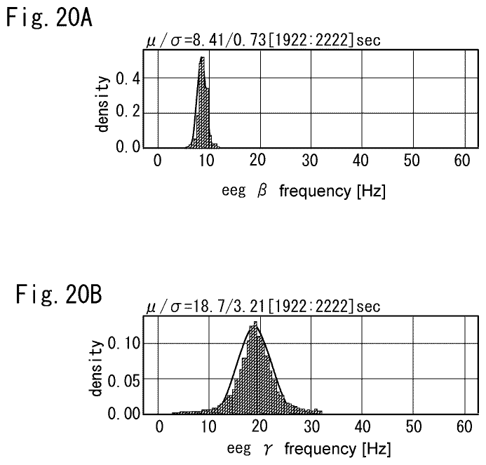

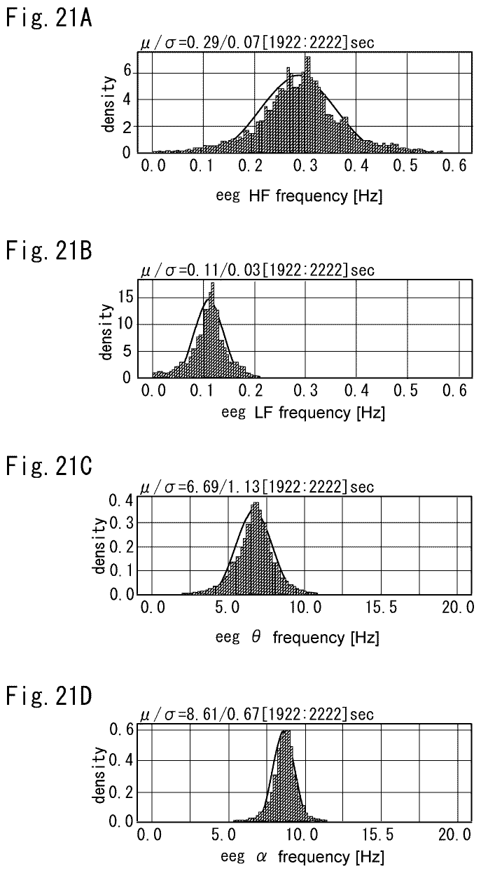

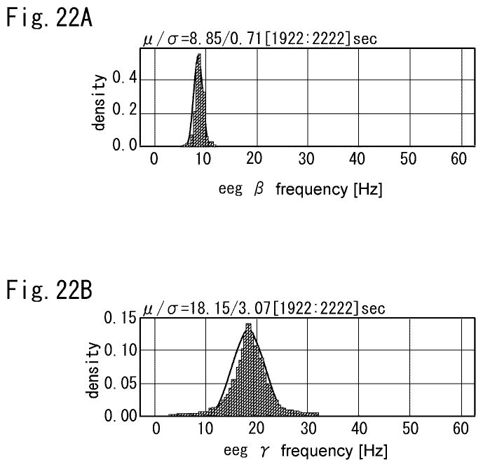

FIGS. 19A-19D and 20A-20B are diagrams illustrating probability density distributions and Gaussian distributions of instantaneous frequencies in brain waves (frontal lobe) of the left brain during rest with eyes closed according to the first embodiment. FIG. 19A shows the HF, FIG. 19B shows the LF, FIG. 19C shows the θ waves, and FIG. 19D shows the α waves. FIG. 20A shows the β waves and FIG. 20B shows the γ waves. FIGS. 21A-21D and 22A-22B are diagrams illustrating a probability density distribution and a Gaussian distribution of an instantaneous frequency in brain waves of the right brain during rest with eyes closed according to the first embodiment. FIG. 21A shows the HF, FIG. 21B shows the LF, FIG. 21C shows the θ waves, and FIG. 21D shows the α waves. FIG. 22A shows the β waves and FIG. 22B shows the γ waves.

As shown in FIGS. 15A-15D, 16A-16B, 17A-17D, and 18A-18B, when the eyes are open and at rest, a subject is generally awake, which means he/she is in a state of reduced parasympathetic activity. Therefore, when the eyes are open and at rest, the subject is in an energetic state. For example, as shown in FIGS. 15C and 17C, the central frequency of the θ waves when the eyes are open and at rest is shifted to a frequency lower than the central frequency during rest with eyes closed shown in FIGS. 19C and 21C. Further, as shown in FIGS. 15D and 17D, the central frequency of the α waves when the eyes are open and at rest is shifted to a frequency higher than the central frequency during rest with eyes closed shown in FIGS. 19D and 21D.

On the other hand, as shown in FIGS. 19A-19D, 20A-20B, 21A-21D, and 22A-22B, when the eyes are closed and at rest, a subject is generally in a state of rest, which means he/she is in a state of improved parasympathetic activity. Therefore, when the eyes are closed and at rest, the subject is relaxed. For example, as shown in FIGS. 19C and 21C, the central frequency of the θ waves during rest with eyes closed is shifted to a frequency higher than the central frequency when the eyes are open and at rest shown in FIGS. 15C and 17C. Further, as shown in FIGS. 19D and 21D, the central frequency of the α waves during rest with eyes closed is shifted to a frequency lower than the central frequency when the eyes are open and at rest shown in FIGS. 15D and 17D.

### <Calculation of Oscillation Waves>

Next, the physiological state control apparatus 50 examines the differences in the feature amounts of the distribution in each band between a time when dysautonomia occurs and a time when the physiological state is normal, and calculates oscillation waves in such a way that the distribution becomes closer to the normal distribution. For example, it is assumed that, due to dysautonomia, the mean of the amplitude distribution in a certain band of the pulse waves in the right internal carotid artery has been changed from M0 to M1, the variance of the amplitude distribution therein from S0 to S1, the mean of the frequency distribution therein has been changed from m0 to m1, and the variance therein from s0 to s1. At this time, if parts where there are modulations in the distributions of amplitudes and frequencies in that band in the brain waves have been found in the right temporal lobe, parietal lobe, and frontal lobe, the oscillator 31 is applied to the right internal carotid artery while monitoring the degree of recovery of the modulations, and an oscillation in which the mean of the distribution of the amplitude is made to approach from M1 to M0, the variance thereof is made to approach from S1 to S0, the mean of the frequency is made to approach from m1 to m0, and the variance thereof is made to approach from s1 to s0 is applied. Accordingly, the symptoms of dysautonomia can be improved. The reason for not abruptly giving the oscillation distribution in the normal physiological state is to avoid applying stress to the living body. The same holds true for a case where the system of the artery flowing into the brain is different or a frequency band in which a modulation is found is different. The oscillation waves may be the ones that are superimposed on one another for each frequency band (k=1, 2, 3, 4 . . . ). The oscillation wave calculation unit 56 may calculate the amplitude and the central frequency of the oscillation waves of a predetermined Gaussian distribution as the amplitude and the central frequency of oscillation waves to be imparted to the living body.

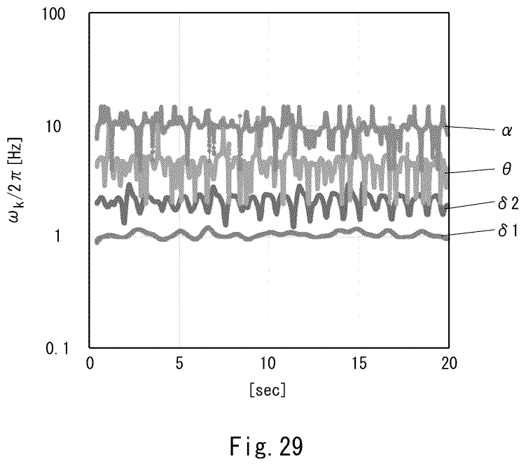

FIGS. 28 and 29 show samples of time-series data of the pulse wave instantaneous amplitude and the pulse wave instantaneous frequency for each band in the normal physiological state. The pulse wave instantaneous amplitude and the instantaneous frequency data are acquired at a time interval in which a Gaussian-like distribution is achieved in a physiologically steady state for each band, and time-series data in which the instantaneous amplitude and the instantaneous frequency continuously change is generated using the above waveforms. At this time, at least second-order time differential values at a timing when the time-series data is started and a timing when it is ended are matched and connected to generate oscillation waveforms in a circular manner using this sample data. When the mean value and the variance value of this sample data are different from desired values, differential addition of the mean values and ratio multiplication of the variance values are performed in such a way that the mean value and the variance value become desired numerical values. When the oscillation waveform is imparted in a plurality of bands, the oscillation waveforms are calculated in each band according to the above-described procedure, a waveform obtained by superimposing them is generated, and the generated waveform is input to the drive apparatus 30.

The oscillation waveform at the normal time may be approximated by a mathematical function to obtain a desired mean value and variance value and input to the drive apparatus 30. In this case, it is required that at least the second-order time differential values be continuous.

If the dysautonomia is relatively mild and the difference from the normal state is small, the oscillation waveform of the band with the difference may be changed to the one in the normal physiological state and then the oscillation waveform in the normal physiological state may be imparted to the oscillator 31 from the beginning.

By adding the calculated oscillation waves to the right and left carotid artery bifurcations or the vertebral arteries branching from the right and left subclavian arteries, the pulse pressure oscillation waves can be put on the cerebral blood flow via the carotid artery or the vertebral artery. Brain activities include activities of the central autonomic nerve system performed in the brainstem in the bands of VLF2 (0.004-0.015 Hz), VLF1 (0.015-0.04 Hz), LF (0.04-0.15 Hz), and HF (0.15-0.4 Hz) and various activities performed in the cerebrum (cortex, limbic system) in each frequency band of δ waves (0.4-4.0 Hz), θ waves (4.0-8.0 Hz), α waves (8.0-13.0 Hz), β waves (13.0-30.0), and γ waves (30-100 Hz). Therefore, the physiological state control system 1 measures brain waves and generates oscillation waves, which are pulse pressure signals, that support brain wave activities in each band. Then, the physiological state control system 1 adds the above-generated oscillation waves from the carotid artery bifurcation to the frontal lobe, the temporal lobe, and the parietal lobe via the internal carotid artery, and from the vertebral artery to the cerebellum and the occipital lobe via the brainstem. Accordingly, the physiological state control system 1 is able to support the brain activities, and support and activate the brain activities in each frequency band.

As one example, the oscillation waves may be calculated from a constant-energy surface of 1/f (it is also referred to as a 1/f spectrum). In the following description, a method for calculating the oscillation waves from the constant-energy surface of 1/f will be described.

### <1/f Constant-Energy Surface>

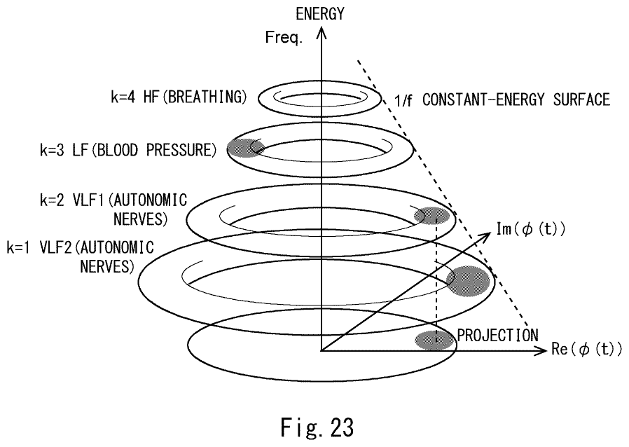

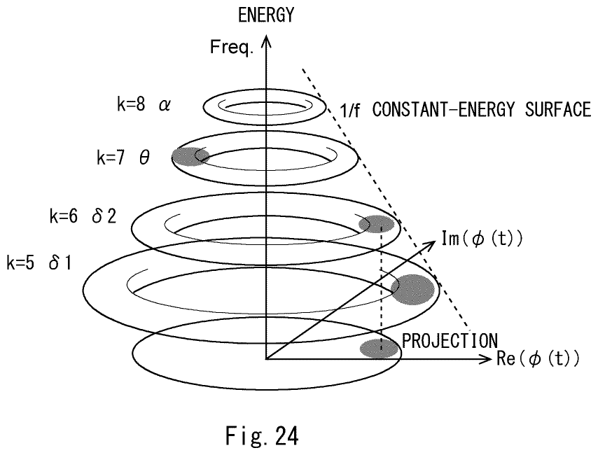

FIGS. 23 and 24 are diagrams each illustrating a complex transformation expression in each frequency band in a three dimensional manner according to the first embodiment, in which the horizontal axis and the vertical axis on a complex plane indicate Re(ln φ(t)) and Im(ln φ(t)) of a complex transformation expression and an axis orthogonal to the complex plane indicates a frequency (energy axis). For example, k=4 in a frequency band longer than the heartbeat interval is expressed as HF (breathing), k=3 is expressed as LF (blood pressure), k=2 is expressed as VLF1 (it may be related to autonomic nerves), and k=1 is expressed as VLF2 (it may be related to autonomic nerves). k=5 in a brain wave band is expressed as δ1 waves, k=6 is expressed as δ2 waves, k=7 is expressed as θ waves, and k=8 is expressed as α waves. Then, the moving radius in the complex plane is a logarithmic amplitude of each frequency band waveform, the energy axis is a frequency axis, and the rotational direction about the energy axis corresponds to a phase angle between the pulse waves and the pulse interval waveform in FIG. 23 and corresponds to the phase angle between the pulse waves and the brain waves in FIG. 24. Then, frequency bands are rotated about the energy axis while they are distributed like a Gaussian distribution in each axis. In this way, a drawing can be obtained in which electrons of each energy level orbit around the nucleus as if they were an electron cloud expressed in terms of the probability of existence. The Gaussian-like distribution of each frequency band, similar to electron clouds, is formed along the constant-energy surface of 1/F.

Accordingly, the oscillation wave calculation unit 56 of the physiological state control apparatus 50 may use, as a predetermined Gaussian distribution, a Gaussian distribution which is along the constant-energy surface of 1/F. That is, the oscillation wave calculation unit 56 may calculate oscillation waves in such a way that the Gaussian distribution of the acquired waveform information becomes the one which is along the constant-energy surface of 1/F.

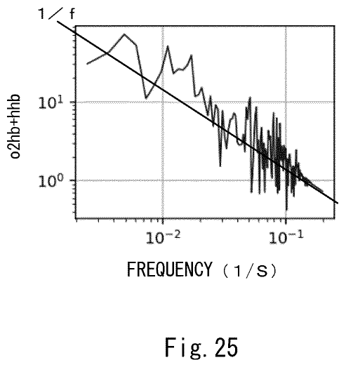

FIG. 25 shows results of measuring the cerebral blood flow by an optical topography technique (NIRS) measured in the frontal lobe. This is a graph illustrating a relation between a total hemoglobin concentration (o2hb+hhb) and a frequency, in which the horizontal axis indicates the frequency and the vertical axis indicates the total hemoglobin concentration. As shown in FIG. 25, the frequency spectrum of the total hemoglobin concentration (o2hb+hhb) is put on the 1/f line. Therefore, it can be considered that a fluctuation spectrum of the amount of the blood flow is put on 1/f.

When an oscillation equal to or smaller than HF (0.4 Hz or smaller) is imparted to blood vessels, the oscillation of the amplitude needs to be larger as the frequency becomes lower. Therefore, it is impossible to implement a procedure that could cause a situation where the blood flow could be stopped by giving such a large amplitude oscillation to the blood vessels. In order to avoid this situation, when an oscillation waveform of a low frequency is put on an oscillation waveform of from about several Hz to 10 Hz after being subjected to AM modulation, it is possible to put a signal of a low frequency on the arterial blood flow without stopping the arterial blood flow.

Further, the physiological state control system 1 may normalize the autonomic nerve system for modulation of physiological states such as intraday and interday variations by performing extension for frequencies lower than VLF2, such as ULF1 (1.5-4 mHz) or ULF2 (0.4-1.5 mHz), like in HF, LF, VLF1 and VLF2, to process waveform information.

The physiological state control system 1 sets, for example, an amplitude and a central frequency for each frequency band of the pulse waves based on a heart rate variability and calculates oscillation waves put on a 1/f spectrum with Gaussian-like fluctuations. Then, by imparting the calculated oscillation waves to the carotid artery bifurcation or the vertebral artery, a pulse pressure oscillation in the 1/f spectrum can be applied to the cerebral arterial flow. Then, by optimizing the oscillation waves while feeding back the pulse waves and the brain waves, the physiological state control system 1 can assist or support the autonomic nerves or brain activities in such a way that the autonomic nerves are regulated or the brain wave activities are activated.

### Second Embodiment

Next, a physiological state control system according to a second embodiment will be described. In the physiological state control system according to this embodiment, only variations of an autonomic nerve system longer than a cardiac cycle are taken into account as waveform information on a living body. Therefore, bands of brain waves equal to or higher than 0.4 Hz are not used and only a pulse wave waveform is used. The pulse wave waveform may be a photoelectric plethysmographic waveform, a piezoelectric waveform obtained from a pulse pressure, a Doppler effect processing waveform reflected on an arterial surface when ultrasound is used, or time-series data of an arterial diameter calculated from a moving image of a cross section of an artery obtained by ultrasound tomography. Since signals in bands of 0.4 Hz or lower are generated by heart rate variability of the heart under the control of the brainstem, the pulse waves can be acquired anywhere in the body. The photoelectric plethysmographic waveforms do not need to be a near-infrared light and may be only a common green light. However, it is necessary to use a signal waveform without the high-pass filter around 0.1 Hz, like a commercially available pulse wave sensor.

### <Waveform Information Acquisition Step>

A waveform information acquisition unit 51 of a physiological state control apparatus 50 acquires waveform information that a communication unit 50b has received from a waveform measurement device such as a pulse wave measurement device 20. The waveform information acquisition unit 51 acquires a pulse wave waveform from time-series data of pulse waves.

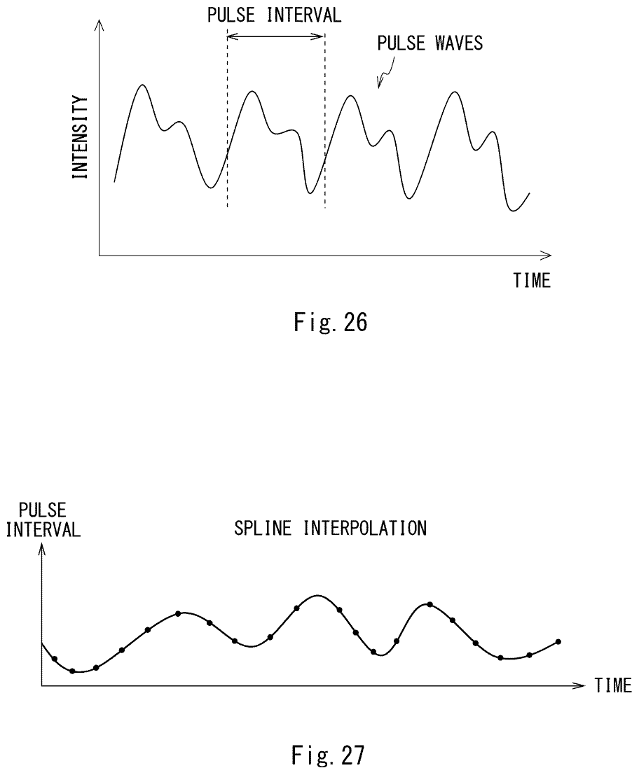

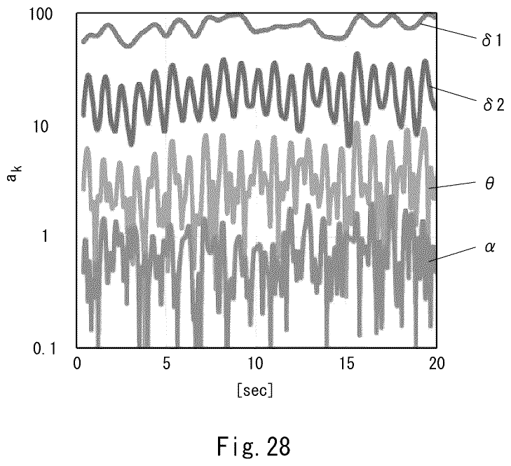

FIG. 26 is a diagram illustrating definitions of a pulse wave waveform and a pulse interval according to the second embodiment. FIG. 27 is a diagram illustrating definitions of pulse interval waves according to the second embodiment. As shown in FIG. 26, pulse interval waves R(t) as shown in FIG. 27 are obtained by calculating a pulse interval (PPI: Peak-Peak Interval) from pulse wave waveform data B(t) using a pulse wave rising position detection algorithm presented in Non-Patent Literature 1, and plotting rising times of the pulse waves and the pulse interval to perform spline interpolation. The pulse interval waves as shown in FIG. 27 may be used, as waveform information, to calculate oscillation waves. That is, the physiological state control apparatus 50 may calculate, as waveform information, oscillation waves to be imparted to the living body based on the pulse interval waves.

### <Filtering Step>

As frequency bands in which a pulse wave waveform and a pulse interval waveform are filtered, VLF2 (0.004-0.015 Hz), VLF1 (0.015-0.04 Hz), LF (0.04-0.15 Hz), and HF (0.15-0.4 Hz) are, for example, selected. VLF1 and VLF2 are fluctuation bands related to the functions of autonomic nerves such as thermoregulation, digestion, excretion, reproduction, immunity, etc., and HF is a respiratory fluctuation band. Further, LF is a blood pressure fluctuation band.

As a result of processing similar to that in the first embodiment, an instantaneous logarithmic amplitude in Equation (9) and an instantaneous frequency in Equation (10) are obtained from pulse wave data. The symbols in the equations according to the second embodiment may indicate components different from those shown in the equations in the first embodiment.

$${a}_{k} ( t ) \qquad ( 9 )$$

$${ω}_{k} ( t ) = d {ψ}_{k} ( t ) / dt$$

From the pulse interval data, an instantaneous logarithmic amplitude in Equation (11) and an instantaneous frequency in Equation (12) can be obtained.

$${A}_{k} ( t ) \qquad ( 11 )$$

$${Ω}_{k} ( t ) = d {Ψ}_{k} ( t ) / dt$$

As an instantaneous phase difference in the time-series data of the pulse interval waveform and the pulse waves, Equation (13) can be obtained.

$${θ}_{k} ( t ) = {Ψ}_{k} ( t ) - {ψ}_{k} ( t ) \qquad ( 13 )$$

As described above, the instantaneous value calculation unit 54 calculates instantaneous values including instantaneous logarithmic amplitudes aₖ(t) and Aₖ(t) corresponding to a real part of an exponential part of a complex waveform equation obtained by performing Hilbert transformation on the waveform information, instantaneous frequencies ωₖ(t) and Ωₖ(t) corresponding to a time differential value of an imaginary part of the exponential part of the complex waveform equation, and a phase difference θₖ(t) between a pulse interval and pulse waves.

### <Distribution Calculation>

Next, the distribution calculation unit 55 of the physiological state control apparatus 50 calculates a distribution of instantaneous values of the pulse waves and the pulse interval waveform. Specifically, the distribution calculation unit 55 calculates probability density distributions of the instantaneous logarithmic amplitudes aₖ(t) and Aₖ(t), the instantaneous frequencies ωₖ(t) and Ωₖ(t), and the instantaneous phase difference θₖ(t) between the pulse waves and the pulse interval at a time interval in which the living body can be regarded as being in a physiologically steady state, causes these probability density distributions to be fit to, for example, a Gaussian distribution to acquire the respective distribution shapes of the instantaneous logarithmic amplitude, the instantaneous frequency, and the instantaneous phase difference, and stores the distribution shapes in the storage unit 50c. Since the value of the instantaneous phase difference is limited to a range of −π to +π, a Von Mises distribution is, for example, applied.

### <Calculation of Oscillation Waves>

Next, the physiological state control apparatus 50 examines the differences in the feature amounts of the distribution in each band between a time when dysautonomia appears and a time when the physiological state is normal, and calculates oscillation waves in such a way that this distribution becomes closer to the normal distribution. For example, it is assumed that, due to dysautonomia, the mean of the amplitude distribution in a certain band of the pulse waves in VLF2 has been changed from M0 to M1, the variance of the amplitude distribution therein from S0 to S1, the mean of the frequency distribution therein has been changed from m0 to m1, and the variance of the frequency distribution therein from s0 to s1. While the variations in the heartbeat interval in bands of 0.4 Hz or smaller are caused by brainstem functions, since the brain waves are not monitored, it is possible to measure the pulse waves at a fingertip or the like to calculate the physiological feature amount in each frequency band. The oscillator 31 is applied to the right and left vertebral arteries which supply blood to the brainstem or to the carotid artery bifurcation which supplies blood to the frontal lobe, temporal lobe, and parietal lobe while monitoring the above numerical values, an oscillation in which the mean of the distribution of the amplitude is made to approach from M1 to M0, the variance thereof is made to approach from S1 to S0, the mean of the frequency is made to approach from m1 to m0, and the variance thereof is made to approach from s1 to s0 is applied. Accordingly, the symptoms of dysautonomia can be improved. The reason for not abruptly giving the oscillation distribution in the normal physiological state is to avoid applying stress to the living body. The same holds true for a case where the system of the artery flowing into the brain is different or a band in which a modulation is found is different. The oscillation waves may be the ones that are superimposed for each frequency band (k=1, 2, 3, 4 . . . ). The oscillation wave calculation unit 56 may calculate the amplitude and the central frequency of the oscillation waves of a predetermined Gaussian distribution as the amplitude and the central frequency of oscillation waves to be imparted to the living body.

When an oscillation equal to or smaller than HF (0.4 Hz or smaller) is imparted to blood vessels, it is possible that the blood flow could be stopped by giving a large amplitude oscillation to the blood vessels. In order to avoid this situation, a signal obtained by performing AM modulation on an oscillation waveform of about several Hz to 10 Hz with a low frequency of HF or lower is input to the drive apparatus, and an oscillation is applied to parts near the vertebral arteries of the right and left cervical vertebrae or parts near the right and left carotid artery bifurcations via an oscillator. Since the vertebral arteries supply blood to the brainstem, it can be expected that a greater effect can be obtained for dysautonomia.

When the dysautonomia is relatively mild and the difference from the normal state is small, the oscillation waveform of the band with the difference may be changed to the one in the normal physiological state and then the oscillation waveform in the normal physiological state may be imparted to the oscillator 31 from the beginning.

Further, the pulse wave measurement sensor 21 needs to detect information on blood vessels located several centimeters deeper from the surface of the living body, use a wavelength from 700 to 2000 nm, which is called an optical window, taking into consideration the absorption spectrum of hemoglobin and water, which are main light-absorbing substances that exist in the living body, and also be able to observe from 0.004 Hz, which is a lower limit of the VLF2 frequency, to 200 Hz, which is twice the upper limit of the brain waves called gamma waves, that is, 100 Hz, considering the Nyquist condition.

Further, the physiological state control system 1 is able to normalize the autonomic nerve system for modulation of physiological states such as intraday or interday variations by performing extension for frequencies lower than VLF2, such as ULF1 (1.5-4 mHz) or ULF2 (0.4-1.5 mHz), like in LF and VLF, to process waveform information.

Note that the present disclosure is not limited to the above-described embodiments and may be changed as appropriate without departing from the spirit of the present disclosure. For example, a combination of configurations of the first and second embodiments is also included in the scope of the technical idea of this embodiment. Further, the following oscillation wave calculation program which causes a computer to execute calculation of oscillation waves performed by the physiological state control apparatus 50 is also included within the scope of the technical idea.

### (Supplementary Note 1)

An oscillation wave calculation program causing a computer to execute:
acquiring waveform information on a living body;
filtering the acquired waveform information in at least one frequency band;
performing Hilbert transformation on the waveform information filtered in the frequency band;
calculating an instantaneous value including at least one of an instantaneous logarithmic amplitude corresponding to an absolute value of a logarithmic amplitude term of a complex waveform equation obtained by performing Hilbert transformation on the waveform information, an instantaneous frequency corresponding to a time differential value of a phase term of a logarithm of the complex waveform equation, and an instantaneous phase corresponding to the phase term of the logarithm of the complex waveform equation;
calculating a Gaussian distribution of the instantaneous value; and
calculating oscillation waves in such a way that the calculated Gaussian distribution becomes a predetermined Gaussian distribution.

### (Supplementary Note 2)

The oscillation wave calculation program according to Supplementary Note 1, wherein
in the filtering, at least one of bands of 0.004-0.015 Hz, 0.015-0.04 Hz, 0.04-0.15 Hz, 0.15-0.4 Hz, 0.4-1.5 Hz, 1.5-4 Hz, 4-15 Hz, and 15-40 Hz is included as the frequency band.

### (Supplementary Note 3)

The oscillation wave calculation program according to Supplementary Note 1, wherein
in the calculation of the oscillation waves,
the oscillation wave are calculated so as to shift a central frequency of a band of θ waves during rest with eyes opened to a frequency lower than the central frequency of the band of the θ waves during rest with eyes closed, and
the oscillation wave are calculated so as to shift the central frequency of the band of α waves during rest with eyes opened to a frequency higher than the central frequency of the band of the α waves during rest with eyes closed.

### (Supplementary Note 4)

The oscillation wave calculation program according to any one of Supplementary Notes 1 to 3, wherein
in the calculation of the oscillation waves,
the oscillation waves to be imparted to the living body are calculated as the waveform information based on at least one of brain waves, pulse waves, or pulse interval waves.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A physiological state control system comprising:
a waveform measurement device configured to acquire waveform information on a living body;
a physiological state control apparatus configured to calculate oscillation waves to be imparted to the living body based on the waveform information acquired by the waveform measurement device; and
a drive apparatus configured to impart the calculated oscillation waves to at least one of a left-side carotid artery bifurcation, a right-side carotid artery bifurcation, a vertebral artery branched out from a left-side subclavian artery, and a vertebral artery branched out from a right-side subclavian artery of the living body from the outside of the living body.

2. The physiological state control system according to claim 1, wherein
the waveform information comprises first waveform information and second waveform information, and
the physiological state control apparatus:
filters, in at least one frequency band, the first waveform information and the second waveform information that have been acquired;
performs Hilbert transformation on the first waveform information and the second waveform information filtered in the frequency band;
calculating an instantaneous value including at least one of an instantaneous logarithmic amplitude corresponding to a real part of a logarithm of a complex waveform equation obtained by performing Hilbert transformation on the first waveform information, an instantaneous logarithmic amplitude corresponding to a real part of a logarithm of a complex waveform equation obtained by performing Hilbert transformation on the second waveform information, an instantaneous frequency corresponding to a time differential value of an imaginary part of the logarithm of the complex waveform equation obtained by performing Hilbert transformation on the first waveform information, an instantaneous frequency corresponding to a time differential value of an imaginary part of the logarithm of the complex waveform equation obtained by performing Hilbert transformation on the second waveform information, and an instantaneous phase difference corresponding to a difference between the imaginary part of the logarithm of the complex waveform equation obtained by performing Hilbert transformation on the first waveform information and the imaginary part of the logarithm of the complex waveform equation obtained by performing Hilbert transformation on the second waveform information;
calculating, during a predetermined period in which the living body can be regarded as being in a physiologically steady state, a probability density distribution of the instantaneous value and approximating the calculated probability density distribution as a predetermined distribution; and
storing a mean value and a variance value of the approximated predetermined distribution as feature amounts of a physiological state.

3. The physiological state control system according to claim 2, wherein the first waveform information is pulse waves and the second waveform information is brain waves, or the first waveform information is pulse waves and the second waveform information is a waveform obtained by performing interpolation resampling on pulse interval data calculated from the pulse waves in a predetermined frequency.

4. The physiological state control system according to claim 2, wherein the physiological state control apparatus includes, as the frequency band, at least one of bands of 0.004-0.015 Hz, 0.015-0.04 Hz, 0.04-0.15 Hz, 0.15-0.4 Hz, 0.4-1.5 Hz, 1.5-4 Hz, 4-8 Hz, 8-13 Hz, 13-30 Hz, and 30-Hz.

5. The physiological state control system according to claim 4, wherein, when the probability density distribution of the instantaneous frequency is a multimodal distribution, the frequency band is divided into a plurality of bands in such a way that the probability density distribution becomes a monomodal distribution.

6. The physiological state control system according to claim 2, wherein the physiological state control apparatus calculates a feature amount of the physiological state in a physiologically normal state and a feature amount of the physiological state in a physiologically abnormal state, which is different from the normal state, compares the feature amounts to detect a difference between them, and calculates the oscillation waves which decrease the difference between them and gradually brings the latter feature amount close to the former feature amount.

7. The physiological state control system according to claim 6, wherein the physiological state control apparatus detects, as the waveform information, a difference between the physiologically normal state and the physiologically abnormal state, which is different from the normal state, based on brain waves, pulse waves, and at least one of a phase difference between the brain waves and the pulse waves or a phase difference between the pulse waves and a pulse interval waveform.

8. The physiological state control system according to claim 1, wherein the oscillation waves are input to the drive apparatus and the calculated oscillation waves are imparted to at least one of the left-side carotid artery bifurcation, the right-side carotid artery bifurcation, the left-side vertebral artery, or the right-side vertebral artery via an oscillator.

9. The physiological state control system according to claim 7, wherein, when the oscillation waves are imparted, the state of the feature amount as it approaches the physiologically normal state is monitored.

10. The physiological state control system according to claim 8, wherein, in a frequency band lower than 0.4 Hz, oscillation waves obtained by performing AM modulation using oscillation waves in a range from 2 Hz to 13 Hz are input to the drive apparatus and imparted to at least one of the left-side carotid artery bifurcation, the right-side carotid artery bifurcation, the left-side vertebral artery, or the right-side vertebral artery via the oscillator.

11. A physiological state control method comprising:
acquiring waveform information on a living body; and
calculating oscillation waves to be imparted to the living body based on the acquired waveform information; and
imparting the calculated oscillation waves to at least one of a left-side carotid artery bifurcation, a right-side carotid artery bifurcation, a vertebral artery branched out from a left-side subclavian artery, and a vertebral artery branched out from a right-side subclavian artery of the living body from the outside of the living body.

12. The physiological state control method according to claim 11, wherein
the calculation of the oscillation wave comprises:
filtering the acquired waveform information in at least one frequency band;
performing Hilbert transformation on the waveform information filtered in the frequency band;
calculating an instantaneous value including at least one of an instantaneous logarithmic amplitude corresponding to a real part of a logarithm of a complex waveform equation obtained by performing Hilbert transformation on the waveform information or an instantaneous frequency corresponding to a time differential value of an imaginary part of the logarithm of the complex waveform equation obtained by performing Hilbert transformation on the waveform information;
calculating a probability density distribution of the instantaneous value during a predetermined period in which the living body can be regarded as being in a physiologically steady state; and
approximating the calculated probability density distribution to a Gaussian distribution; and
calculating the oscillation waves in such a way that the probability density distribution becomes the predetermined Gaussian distribution.

13. The physiological state control method according to claim 12, wherein the probability density distribution is a monomodal distribution including a Gaussian distribution.

14. The physiological state control method according to claim 12, wherein
in the acquisition of the waveform information on the living body,
pulse waves and brain waves are acquired as the waveform information, the calculation of the oscillation wave comprises:
filtering, in at least one frequency band, the acquired pulse wave, the acquired brain wave, and a pulse interval waveform,
performing Hilbert transformation on the pulse waves, the brain waves, and the pulse interval waveform filtered in the frequency band,
calculating an instantaneous value including at least one of an instantaneous logarithmic amplitude, which is a real part of a logarithm of a complex waveform equation obtained by performing Hilbert transformation on the pulse waves, the brain waves, and the pulse interval waveform, an instantaneous frequency, which is a time differential value of a phase, which is an imaginary part of the logarithm of the complex waveform equation obtained by performing Hilbert transformation on the pulse waves, the brain waves, and the pulse interval waveform, an instantaneous phase difference corresponding to a difference between a phase, which is an imaginary part of a logarithm of a complex waveform equation obtained by performing Hilbert transformation on the brain waves, and a phase, which is an imaginary part of a logarithm of a complex waveform equation obtained by performing Hilbert transformation on the pulse waves, and an instantaneous phase difference corresponding to a difference between a phase, which is an imaginary part of a logarithm of a complex waveform equation obtained by performing Hilbert transformation on the pulse interval waveform, and a phase, which is an imaginary part of a logarithm of a complex waveform equation obtained by performing Hilbert transformation on the pulse waves;
calculating the probability density distribution of the instantaneous value during a predetermined period in which the living body can be regarded as being in a physiologically steady state; and
calculating the oscillation waves in such a way that the calculated probability density distribution becomes a predetermined distribution.

15. The physiological state control method according to claim 12, wherein
in the acquisition of the waveform information on the living body,
the pulse waves are acquired by a photoplethysmogram which measures pulse waves flowing into a brain, and
a wavelength of a light to be used is in a range from 700 to 2000 nm and a frequency bandwidth is in a range from 0.004 Hz to 200 Hz.

16. A non-transitory computer readable medium storing an oscillation wave calculation program for causing a computer to execute:
acquiring waveform information on a living body;
filtering the acquired waveform information in at least one frequency band;
performing Hilbert transformation on the waveform information filtered in the frequency band;
calculating an instantaneous value including at least one of an instantaneous logarithmic amplitude corresponding to an absolute value of a logarithmic amplitude term of a complex waveform equation obtained by performing Hilbert transformation on the waveform information, an instantaneous frequency corresponding to a time differential value of a phase term of a logarithm of the complex waveform equation, and an instantaneous phase corresponding to the phase term of the logarithm of the complex waveform equation;
calculating a Gaussian distribution of the instantaneous value; and
calculating oscillation waves in such a way that the calculated Gaussian distribution becomes a predetermined Gaussian distribution.

17. The non-transitory computer readable medium storing the oscillation wave calculation program according to claim 16, wherein
in the filtering, at least one of bands of 0.004-0.015 Hz, 0.015-0.04 Hz, 0.04-0.15 Hz, 0.15-0.4 Hz, 0.4-1.5 Hz, 1.5-4 Hz, 4-15 Hz, and 15-40 Hz is included as the frequency band.

18. The non-transitory computer readable medium storing the oscillation wave calculation program according to claim 16, wherein
in the calculation of the oscillation waves,
the oscillation wave are calculated so as to shift a central frequency of a band of θ waves during rest with eyes opened to a frequency lower than the central frequency of the band of the θ waves during rest with eyes closed, and
the oscillation wave are calculated so as to shift the central frequency of the band of a waves during rest with eyes opened to a frequency higher than the central frequency of the band of the α waves during rest with eyes closed.

19. The non-transitory computer readable medium storing the oscillation wave calculation program according to claim 16, wherein
in the calculation of the oscillation waves,
the oscillation waves to be imparted to the living body are calculated as the waveform information based on at least one of brain waves, pulse waves, or pulse interval waves.
